(19) United States
(12) Patent Application Publication
(10) Pub. No.: US 2022/0073986 A1
(43) Pub. Date: Mar. 10, 2022

(54) METHOD OF CHARACTERIZING A NEURODEGENERATIVE PATHOLOGY

(71) Applicant: VIVID GENOMICS, INC., San Diego, CA (US)

(72) Inventors: Michael NALLS, San Diego, CA (US); Marcel VAN DER BRUG, San Diego, CA (US); Julie COLLENS, San Diego, CA (US); IIya CHORNY, San Diego, CA (US)

(21) Appl. No.: 17/276,339

(22) PCT Filed: Sep. 17, 2019
(86) PCT No.: PCT/US2019/051547

§ 371 Date: Mar. 15, 2021

Related U.S. Application Data
(60) Provisional application No. 62/732,883, filed on Sep. 18, 2018.
(60) Provisional application No. 62/783,982, filed on Dec. 21, 2018.

(51) Int. Cl. C12Q 1/6883; G16B 40/00; G16H 50/20

(52) U.S. Cl. CPC C12Q 1/6883; G16B 40/00; C12Q 2600/118; C12Q 2600/156; G16H 50/20

(57) ABSTRACT

Provided herein is technology relating to detecting and/or identifying cognitive impairment in a subject and particularly, but not exclusively, to compositions, methods, systems, and kits for identifying individuals who have cognitive impairment or who have an increased risk of having cognitive impairment.

### CROSS-REFERENCE TO RELATED APPLICATIONS

This application claims priority benefit of U.S. Provisional Application No. 62/732,883, filed on Sep. 18, 2018; and U.S. Provisional Application No. 62/783,982, filed on Dec. 21, 2018; each of which is incorporated herein by reference in its entirety for all purposes.

### FIELD

Provided herein is technology relating to detecting and/or identifying cognitive impairment in a subject and particularly, but not exclusively, to compositions, methods, systems, and kits for identifying individuals who have cognitive impairment or who have an increased risk of having cognitive impairment. Also provided are methods for characterizing risk of a cognitive impairment or one or more neurodegenerative pathological features associated with a neurodegenerative pathological feature, and methods of selecting subjects for a clinical trial based on the characterized risk.

### BACKGROUND

Brain pathology is relevant to clinical trials of neurodegenerative diseases and other diseases of cognitive impairment. However, pre-mortem brain pathology data are often difficult (e.g., in terms of accessibility or feasibility) or costly (e.g., in terms of time or money) or not possible (e.g., Lewy bodies) to obtain. In particular, most pre-mortem brain pathology data are acquired using imaging technologies or invasive sampling, which are disadvantageous because they are not portable, have high material costs, and are often uncomfortable for patients.

### SUMMARY

Accordingly, provided herein is technology for identifying and/or classifying cognitive impairment. In some embodiments, the technology relates to inferring the presence of neurodegenerative pathological features (e.g., inferring the presence of tau protein, amyloid beta, cerebral amyloid angiopathy (CAA) and/or Lewy bodies) in a patient using genomic data and, optionally, clinical data and/or therapeutic data. In some embodiments, genomic data, clinical data, and/or therapeutic data are used as inputs to a machine (e.g., deep) learning framework that combines disparate predictive paths or trees into an aggregate predictor and/or classifier of cognitive impairment for a patient. In some embodiments, the genomic data comprises genotype data, haplotype data, genotypic variation data, haplotypic variation data, polymorphism (e.g., single nucleotide polymorphism) data, and/or genotypes tagging haplotypic variation. In some embodiments, the genomic data comprises known risk loci for neurodegenerative diseases or a locus in linkage disequilibrium with known risk loci for neurodegenerative diseases. The predictor and/or classifier is/are based on a nonlinear combination of data and thus provides a more powerful predictor than existing linear polygenic genetic risk score methods.

In some embodiments, the technology relates to identifying genomic data, clinical data, and/or therapeutic data from reference samples that are pathologically characterized, e.g., neural tissue (e.g., brain) samples known to comprise neurodegenerative pathological features (e.g., tau protein, amyloid beta, cerebral amyloid angiopathy (CAA), and/or Lewy bodies). In some embodiments, machine (e.g., deep) learning technologies are used to build clinico-genetic models that predict the incidence and quantity of the neurodegenerative pathological features (e.g., tau protein, amyloid beta, cerebral amyloid angiopathy (CAA) and/or Lewy bodies) in the reference samples.

Provided herein is a method for characterizing a plurality of neurodegenerative pathological features of a cognitive impairment in a human subject, comprising: (a) detecting, in a sample obtained from the subject, a status of first markers in a first panel of markers or markers in linkage disequilibrium with markers in the first panel of markers, wherein the first panel of markers is associated with a first neurodegenerative pathological feature of the cognitive impairment; (b) detecting, in the same sample obtained from the subject, a status of second markers in a second panel of markers or markers in linkage disequilibrium with markers in the second panel of markers, wherein the second panel of markers is associated with a second neurodegenerative pathological feature of the cognitive impairment; and (c) characterizing a presence or risk the first and second neurodegenerative pathological features of the cognitive impairment in the subject based on the status of the first markers and the status of the second markers. In some embodiments, detecting a status of first markers or a status of second markers comprises determining the presences or absence of the first markers or the presence or absence of the second markers. In some embodiments, the presence or risk of the first neurodegenerative pathological feature and the presence or risk of the second neurodegenerative pathological feature are characterized using independently selected machine learning systems. In some embodiments, the method comprises characterizing a presence or risk of three or more neurodegenerative pathological features of the cognitive impairment in the subject using independently selected machine learning systems. In some embodiments, the first neurodegenerative pathological feature and/or the second neurodegenerative pathological feature is amyloid beta, Lewy bodies, tau protein, cerebral amyloid angiopathy (CAA), or a progression of the cognitive impairment. In some embodiments, the first markers and/or the second markers comprise one or more genetic markers. In some embodiments, the one or more genetic markers comprise one or more functional SNPs and/or one or more tag SNPs. In some embodiments, the one or genetic markers comprise one or more of a DNA structural variant, a DNA copy number, a DNA repeat expansion, a DNA short tandem repeat (SIR), DNA deletion 20 bases in length or less, a DNA deletion more than 21 bases in length, a DNA insertion, an RNA expression level, an RNA SNP, an RNA fusion, an RNA splice variant, or a DNA methylation status. In some embodiments, detecting the status of the genetic marker comprises determining an identity of a nucleotide at a chromosomal location of the genetic marker. In some embodiments, the first markers and/or the second markers comprise clinical markers and/or therapeutic markers. In some embodiments, said markers comprise an APOE allele 2 copy number, APOE allele 4 copy number, biological sex, and/or age. In some embodiments, characterizing the presence or risk of the first and second neurodegenerative pathological features of the cognitive impairment in the subject comprises inputting data describing the status of the first set of markers and/or the second set of markers into one or more machine learning systems. In some embodiments, the one or more machine learning systems output a predictor of the presence or risk of the first neurodegenerative pathological feature and the presence or risk of the second neurodegenerative pathological feature. In some embodiments, at least the first neurodegenerative pathological feature and the second neurodegenerative pathological feature are used to enroll the subject in a clinical trial. In some embodiments, at least the first neurodegenerative pathological feature and the second neurodegenerative pathological feature are used to determine a course of a treatment for the cognitive impairment. In some embodiments, detecting the status of one or more markers among the first markers or the second markers comprises use of a detection technique selected from the group consisting of microarray analysis, nucleic acid amplification, hybridization analysis, and next generation sequencing. In some embodiments, detecting the status of one or more markers among the first markers or the second markers comprises sequencing nucleic acids from the sample.

Also provided herein is a method for characterizing a human subject as having a cognitive impairment, the method comprising detecting, in a sample obtained from the subject, the presence or absence of markers for a panel of markers or markers in linkage disequilibrium with the markers; and characterizing the presence or risk of cognitive impairment in the subject based on the presence or absence of said markers of said panel of markers. In some embodiments, the human subject is suspected of suffering from a cognitive disorder based on the presence of symptoms of a cognitive disorder. In some embodiments, the human subject is suspected of suffering from a cognitive disorder based on an assessment of cognitive ability (e.g., MMSE, CDR-SB), In some embodiments, the human subject is suspected of suffering from a cognitive disorder based on a change with time of a score from an assessment of cognitive ability (e.g., MMSE, CDR-SB).

Also provided herein is a method for characterizing a human subject as having a cognitive impairment, the method comprising detecting, in a sample obtained from the subject, the presence or absence of markers for a panel of markers selected from the markers provided by Table 2 or markers in linkage disequilibrium with the markers in Table 2; and characterizing the presence or risk of cognitive impairment in the subject based on the presence or absence of said markers of said panel of markers. In some embodiments, the human subject is suspected of suffering from a cognitive disorder based on the presence of symptoms of a cognitive disorder. In some embodiments, the human subject is suspected of suffering from a cognitive disorder based on an assessment of cognitive ability (e.g., MMSE, CDR-SB). In some embodiments, the human subject is suspected of suffering from a cognitive disorder based on a change with time of a score from an assessment of cognitive ability (e.g., MMSE, CDR-SB).

In some embodiments, characterizing the presence or risk of cognitive impairment in the subject comprises inputting data describing the presence or absence of said markers of said panel of markers into a machine learning system. In some embodiments, characterizing the presence or risk of cognitive impairment in the subject further comprises inputting data describing clinical and/or therapeutic markers into said machine learning system. In some embodiments, the clinical and/or therapeutic markers comprise a marker selected from the group consisting of APOE allele 4 copy number, APOE allele 2 copy number, biological sex, and age. In some embodiments, the machine learning system outputs a predictor of cognitive impairment in the subject. In some embodiments, the markers of said panel of markers comprise functional SNPs and/or tag SNPs. In some embodiments, detecting the presence or absence of a marker in the panel of markers comprises determining the identity of a nucleotide at the chromosomal location of said marker. In some embodiments, detecting the presence or absence of a marker in the panel of markers comprises exposing the sample to nucleic acid probes complementary to the genomic sequences corresponding to the markers of the panel. In some embodiments, the nucleic acid probes are covalently linked to a solid surface. In some embodiments, detecting the presence or absence of a marker in the panel of markers comprises use of a detection technique selected from the group consisting of microarray analysis, nucleic acid amplification, and hybridization analysis. In some embodiments, detecting the presence or absence of a marker in the panel of markers comprises sequencing nucleic acids from the sample.

In some embodiments, the panel of markers comprises 5 markers, 10 markers. 20 markers, 50 markers, or more than 50 markers. In some embodiments, the panel comprises 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 or more markers.

In some embodiments, the technology provides a method for classifying progression of cognitive impairment in a human subject, the method comprising detecting, in a sample obtained from the subject, the presence or absence of markers for a panel of markers or markers in linkage disequilibrium with the markers; and classifying progression of cognitive impairment in the human subject based on the presence or absence of said markers of said panel of markers. In some embodiments, the human subject is suspected of suffering from a cognitive disorder based on the presence of symptoms of a cognitive disorder. In some embodiments, the human subject is suspected of suffering from a cognitive disorder based on an assessment of cognitive ability (e.g., MMSE, CDR-SB). In some embodiments, the human subject is suspected of suffering from a cognitive disorder based on a change with time of a score from an assessment of cognitive ability (e.g., MMSE, CDR-SB).

In some embodiments, the technology provides a method for classifying progression of cognitive impairment in a human subject, the method comprising detecting, in a sample obtained from the subject, the presence or absence of markers for a panel of markers selected from the markers provided by Table 1 or markers in linkage disequilibrium with the markers in Table 1; and classifying progression of cognitive impairment in the human subject based on the presence or absence of said markers of said panel of markers. In some embodiments, the human subject is suspected of suffering from a cognitive disorder based on the presence of symptoms of a cognitive disorder. In some embodiments, the human subject is suspected of suffering from a cognitive disorder based on an assessment of cognitive ability (e.g., MMSE, CDR-SB). In some embodiments, the human subject is suspected of suffering from a cognitive disorder based on a change with time of a score from an assessment of cognitive ability (e.g., MMSE, CDR-SB).

In some embodiments, classifying progression of cognitive impairment in said human subject comprises inputting data describing the presence or absence of said markers of said panel of markers into a machine learning system. In some embodiments, classifying progression of cognitive impairment in said human subject further comprises inputting data describing clinical and/or therapeutic markers into said machine learning system. In some embodiments, the clinical and/or therapeutic markers comprise a marker selected from the group consisting of APOE allele 4 copy number, APOE allele 2 copy number, biological sex, and age. In some embodiments, the machine learning system outputs a classifier of progression of cognitive impairment in a human subject. In some embodiments, the markers of said panel of markers comprises functional SNPs and/or tag SNPs. In some embodiments, detecting the presence or absence of a marker in the panel of markers comprises determining the identity of a nucleotide at the chromosomal location of said marker. In some embodiments, detecting the presence or absence of a marker in the panel of markers comprises exposing the sample to nucleic acid probes complementary to the genomic sequences corresponding to the markers of the panel. In some embodiments, the nucleic acid probes are covalently linked to a solid surface. In some embodiments, detecting the presence or absence of a marker in the panel of markers comprises use of a detection technique selected from the group consisting of microarray analysis, nucleic acid amplification, and hybridization analysis. In some embodiments, detecting the presence or absence of a marker in the panel of markers comprises sequencing nucleic acids from the sample.

In some embodiments, the panel of markers comprises 5 markers, 10 markers, 20 markers, 50 markers, or more than 50 markers. In some embodiments, the panel comprises 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 or more markers.

Some embodiments of the technology relate to kits, reagent mixtures, or a surface (e.g., an array). In some embodiments, the technology provides a kit, reagent mixture, or surface comprising reagents for detecting a panel comprising multiple markers from a panel of markers or markers in linkage disequilibrium with a panel of markers. In some embodiments, the kit, reagent mixture, or surface comprises reagents for detection of 1000 or fewer markers. In some embodiments, the kit, reagent mixture, or surface comprises reagents for detection of 5 markers, 10 markers. 20 markers, 50 markers, or more than 50 markers. In some embodiments, the kit, reagent mixture, or surface comprises reagents for detection of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 or more markers.

Some embodiments of the technology relate to kits, reagent mixtures, or a surface (e.g., an array). In some embodiments, the technology provides a kit, reagent mixture, or surface comprising reagents for detecting a panel comprising multiple markers listed in Table 1 or Table 2 or markers in linkage disequilibrium with markers listed in Table 1 or Table 2. In some embodiments, the kit, reagent mixture, or surface comprises reagents for detection of 1000 or fewer markers. In some embodiments, the kit, reagent mixture, or surface comprises reagents for detection of 5 markers, 10 markers, 20 markers, 50 markers, or more than 50 markers. In some embodiments, the kit, reagent mixture, or surface comprises reagents for detection of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 or more markers.

Some embodiments provide a method for characterizing a sample as having been obtained from a human subject having cognitive impairment, the method comprising receiving a sample obtained from the subject; detecting, in a sample obtained from the subject, the presence or absence of a first marker of cognitive impairment selected from the markers provided by Table 2 or in linkage disequilibrium with a marker provided by Table 2; detecting, in said sample, the presence or absence of a second marker of cognitive impairment selected from the markers provided by Table 2 or in linkage disequilibrium with a marker provided by Table 2; using a machine learning system to receive data generated in steps (b) and (c) and output a cognitive impairment risk assessment for the human subject from which the sample was obtained; and generating a report characterizing the sample as having been obtained from a human subject having cognitive impairment or having an increased risk of cognitive impairment based on the risk assessment of step (d). In some embodiments, the methods further co rise identifying said subject as a candidate for a clinical trial.

Some embodiments characterizing the presence or risk of a cognitive impairment comprises predicting the presence of more than one pathological feature, where in each pathological feature has a unique set of panel markers.

Some embodiments provide a method for classifying progression of cognitive impairment in a human subject, the method comprising (a) receiving a sample obtained from the subject; (h) detecting, in a sample obtained from the subject, the presence or absence of one or more markers of cognitive impairment selected from a panel of markers or in linkage disequilibrium with a marker selected from the panel of markers; (c) using a machine learning system to receive data generated in step (b) and output a cognitive impairment progression classifier for the human subject from which the sample was obtained; and (d) generating and/or displaying a report classifying the progression of cognitive impairment in the human subject based on the risk assessment of step (c). In some embodiments, the methods further comprise identifying said subject as a candidate for a clinical trial or for treatment with a particular therapy. In some embodiments, methods are provided that further comprise the step of administering the therapy.

Some embodiments provide a method for classifying progression of cognitive impairment in a human subject, the method comprising (a) receiving a sample obtained from the subject; (b) detecting, in a sample obtained from the subject, the presence or absence of a first marker of cognitive impairment selected from the markers provided by Table 1 or in linkage disequilibrium with a marker provided by Table 1; (c) detecting, in said sample, the presence or absence of a second marker of cognitive impairment selected from the markers provided by Table 1 or in linkage disequilibrium with a marker provided by Table 1; (d) using a machine learning system to receive data generated in step (h) and output a cognitive impairment progression classifier for the human subject from which the sample was obtained; and (e) generating and/or displaying a report classifying the progression of cognitive impairment in the human subject based on the risk assessment of step (d). In some embodiments, the methods further comprise identifying said subject as a candidate for a clinical trial or for treatment with a particular therapy. In some embodiments, methods are provided that further comprise the step of administering the therapy.

In some embodiments, methods are provided for testing a subject for cognitive impairment, the method comprising obtaining a sample from the subject; providing the sample to testing facility to be tested for the presence or absence of markers for a panel of markers or markers in linkage disequilibrium with the markers in the panel of markers; and receiving a report from the testing facility indicating presence or risk of cognitive impairment in the subject.

In some embodiments, methods are provided for testing a subject for cognitive impairment, the method comprising obtaining a sample from the subject; providing the sample to testing facility to be tested for the presence or absence of markers for a panel of markers selected from the markers provided by Table 2 or markers in linkage disequilibrium with the markers in Table 2; and receiving a report from the testing facility indicating presence or risk of cognitive impairment in the subject.

In some embodiments, methods are provided for classifying progression of cognitive impairment in a human subject, the method comprising obtaining a sample from the subject; providing the sample to testing facility to be tested for the presence or absence of markers for a panel of markers or markers in linkage disequilibrium with the markers; and receiving a report from the testing facility classifying progression of cognitive impairment in the human subject.

In some embodiments, methods are provided for classifying progression of cognitive impairment in a human subject, the method comprising obtaining a sample from the subject; providing the sample to testing facility to be tested for the presence or absence of markers for a panel of markers selected from the markers provided by Table 1 or markers in linkage disequilibrium with the markers in Table 1; and receiving a report from the testing facility classifying progression of cognitive impairment in the human subject.

Further embodiments relate to uses of a marker panel comprising markers provided by Table 2 or markers in linkage disequilibrium with the markers in Table 2 to test a subject for cognitive impairment. Further embodiments relate to uses of a marker panel comprising markers provided by Table 1 or markers in linkage disequilibrium with the markers in Table 1 to classify progression of cognitive impairment in a human subject.

In some embodiments the panel of markers comprises DNA copy number variants, DNA repeat expansions, DNA STRs (short tandem repeats), small deletions, large deletions, RNA expression, microRNAs, RNA SNPs, RNA fusions, and DNA methylation status.

In some embodiments tests for multiple neurodegenerative pathological features are used to classify patients for clinical trials.

Additional embodiments will be apparent to persons skilled in the relevant art based on the teachings contained herein.

### BRIEF DESCRIPTION OF THE DRAWINGS

These and other features, aspects, and advantages of the present technology will become better understood with regard to the following drawings:

FIG. 1 is a schematic showing the production of a validated pathology predictor using machine learning and reference samples as described herein.

FIG. 2 is a flowchart showing a method for identifying a subject for enrollment in a clinical trial according to embodiments of the technology described herein.

FIG. 3 shows the ROC describing the performance of an embodiment of a machine learning predictor/classifier according to the technology described herein.

It is to be understood that the figures are not necessarily drawn to scale, nor are the objects in the figures necessarily drawn to scale in relationship to one another. The figures are depictions that are intended to bring clarity and understanding to various embodiments of apparatuses, systems, and methods disclosed herein. Wherever possible, the same reference numbers will be used throughout the drawings to refer to the same or like parts. Moreover, it should be appreciated that the drawings are not intended to limit the scope of the present teachings in any way.

### DETAILED DESCRIPTION

Provided herein is technology relating to detecting and/or identifying cognitive impairment in a subject and particularly, but not exclusively, to compositions, methods, systems, and kits for diagnosing individuals who have cognitive impairment or who have increased risk of having cognitive impairment. Also provided are methods for characterizing risk of a cognitive impairment or one or more neurodegenerative pathological features associated with a neurodegenerative pathological feature, and methods of selecting subjects for a clinical trial based on the characterized risk.

As further described herein, biological markers of cognitive impairment (which may be associated with, for example, a neurodegenerative pathology such as Alzheimer's disease or dementia), or neurodegenerative pathological features of a cognitive impairment, detected in a biological sample obtained from a subject can be analyzed to characterize the presence or risk of the cognitive impairment or the neurodegenerative pathological feature. The determined risk may be a contemporaneous risk (i.e., the risk that the subject has the cognitive impairment or one or more neurodegenerative pathological features at the time the sample was obtained from the subject), or may be a prospective risk (i.e., the risk that the subject will develop the cognitive impairment or the one or more neurodegenerative pathological features). Contemporaneous risk determination for certain neurodegenerative pathological features allows for assessment of the patient during the life of the patient, which is often not possible because such pathology analysis requires brain samples unobtainable in a living subject. Prospective risk assessment is also helpful to predict the likelihood that the subject will develop the cognitive impairment and/or one or more pathological features associated with the cognitive impairment.

Risk assessment of cognitive impairment and/or one or more neurodegenerative pathological features as described herein is useful in selecting or enrolling a patient in a clinical trial, such as a clinical study directed to further understanding cognitive impairment or treatment of cognitive impairment. For example, a clinical study investigating methods to prevent or limit the development of a cognitive impairment may want to enroll a larger proportion of subjects susceptible (i.e., at a high risk) to developing a cognitive impairment and/or one or more neurodegenerative pathological features compared to a general patient population. This helps ensure a sufficiently large number of positive incidences of the cognitive impairment and/or one or more neurodegenerative pathological features and can results in a smaller study cohort, thereby reducing the number treatment-related adverse events and overall cost of the clinical study.

Joint risk of assessment two or more neurodegenerative pathological features associated with cognitive impairment, as a separate risk and/or a composite risk, is also useful, including in selecting and/or enrolling a subject in a clinical trial. Separate risk assessments includes the separate characterization of two or more neurodegenerative pathological features. For example, the risk that a subject has (e.g., at the time a sample was obtained from the subject) or will develop a first neurodegenerative pathological feature may be separately characterized or considered (e.g., for selection and/or enrollment of the subject in a clinical trial) from the risk that the subject has or will develop a second neurodegenerative pathological feature. A composite risk characterization examines the risk that the subject has or will develop one or more of the first neurodegenerative pathological feature and the second neurodegenerative pathological feature (or more, if the risk of additional neurodegenerative pathological features is characterized), or that the subject has or will develop both the first neurodegenerative pathological feature and the second neurodegenerative pathological feature (or more, if the risk of additional neurodegenerative pathological features is characterized). For some purposes, the exact comorbidity is less important than knowing that overall the patient has a high risk of cognitive impairment. In this detailed description of the various embodiments, for purposes of explanation, numerous specific details are set forth to provide a thorough understanding of the embodiments disclosed. One skilled in the art will appreciate, however, that these various embodiments may be practiced with or without these specific details. In other instances, structures and devices are shown in block diagram form. Furthermore, one skilled in the art can readily appreciate that the specific sequences in which methods are presented and performed are illustrative and it is contemplated that the sequences can be varied and still remain within the spirit and scope of the various embodiments disclosed herein.

All literature and similar materials cited in this application, including but not limited to, patents, patent applications, articles, books, treatises, and interact web pages are expressly incorporated by reference in their entirety for any purpose. Unless defined otherwise, all technical and scientific terms used herein have the same meaning as is commonly understood by one of ordinary skill in the art to which the various embodiments described herein belongs. When definitions of terms in incorporated references appear to differ from the definitions provided in the present teachings, the definition provided in the present teachings shall control. The section headings used herein are for organizational purposes only and are not to be construed as limiting the described subject matter in any way.

### Definitions

To facilitate an understanding of the present technology, a number of terms and phrases are defined below. Additional definitions are set forth throughout the detailed description.

Throughout the specification and claims, the following terms take the meanings explicitly associated herein, unless the context clearly dictates otherwise. The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the technology may be readily combined, without departing from the scope or spirit of the technology.

In addition, as used herein, the term “or” is an inclusive “or” operator and is equivalent to the term “and/or” unless the context clearly dictates otherwise. The term “based on” is not exclusive and allows for being based on additional factors not described, unless the context clearly dictates otherwise. In addition, throughout the specification, the meaning of “a”, “an”, and “the” include plural references. The meaning of “in” includes “in” and “on.”

As used herein, the terms “about”, “approximately”, “substantially”, and “significantly” are understood by persons of ordinary skill in the art and will vary to some extent on the context in which they are used. If there are uses of these terms that are not clear to persons of ordinary skill in the art given the context in which they are used, “about” and “approximately” mean plus or minus less than or equal to 10% of the particular term and “substantially” and “significantly” mean plus or minus greater than 10% of the particular term.

As used herein, the suffix “-free” refers to an embodiment of the technology that omits the feature of the base root of the word to which “-free” is appended. That is, the term “X-free” as used herein means “without X”, where X is a feature of the technology omitted in the “X-free” technology. For example, a “calcium-free” composition does not comprise calcium, a “mixing-free” method does not comprise a mixing step, etc.

As used herein, a “pathological marker of neurodegeneration” refers to a marker associated with neurodegeneration, tau protein, amyloid beta, and/or Lewy bodies.

As used herein, a “positive” sample refers to a sample comprising a pathological marker of neurodegeneration and that reports a predictor value above a threshold value (e.g., the range associated with disease). As used herein, a “positive” subject refers to a subject having cognitive impairment (e.g., as indicated by an assessment of cognitive skills or cognitive impairment (e.g., Mini-Mental State Exam (MMSE)) and that reports a predictor value above a threshold value (e.g., the range associated with disease of) or the Clinical Dementia Rating Scale Sum of Boxes (CDR-SB)). As used herein, a “false negative” refers to a positive sample or a positive subject that reports a predictor value below the threshold value (e.g., the range associated with no disease).

As used herein, a “negative” sample refers to a sample that does not comprise a pathological marker of neurodegeneration or in which a pathological marker of neurodegeneration is not detectable and that reports a predictor value below a threshold value (e.g., the range associated with no disease). As used herein, a “negative” subject refers to a subject who does not have cognitive impairment (e.g., as indicated by an assessment of cognitive skills or cognitive impairment (e.g., Mini-Mental State Exam (MMSE)) and that reports a predictor value below a threshold value (e.g., the range associated with no disease). As used herein, a “false positive” refers to a negative sample or negative subject that reports a predictor value above the threshold value (e.g., the range associated with disease).

As used herein, the “sensitivity” of a given predictor refers to: a) the percentage of positive samples that report a predictor value above a threshold value that distinguishes positive samples from negative samples; orb) the percentage of positive subjects that report a predictor value above a threshold value that distinguishes positive subjects from negative subjects. The value of sensitivity, therefore, reflects the probability that a predictor value produced for a known diseased sample or known cognitively impaired subject will be in the range of disease-associated measurements. As defined here, the clinical relevance of the calculated sensitivity value represents an estimation of the probability that a given predictor value would detect the presence of a clinical condition when applied to a subject with that condition or a sample obtained from a subject with that condition.

As used herein, the “specificity” of a given predictor refers to: a) the percentage of negative samples that report a predictor value below a threshold value that distinguishes positive samples from negative samples; or b) the percentage of negative subjects that report a predictor value below a threshold value that distinguishes positive subjects from negative subjects. The value of specificity; therefore, reflects the probability that a predictor value produced for from a known non-diseased sample or non-cognitively impaired subject will be in the range of non-disease associated measurements. As defined here, the clinical relevance of the calculated specificity value represents an estimation of the probability that a given predictor value would detect the absence of a clinical condition when applied to a subject without that condition or to a sample obtained from a subject without that condition.

The term “AUC” as used herein is an abbreviation for the “area under a curve”. In particular it refers to the area under a Receiver Operating Characteristic (ROC) curve. An “ROC curve” is a plot of the true positive rate against the false positive rate for the different possible cut points of a diagnostic test. It shows the trade-off between sensitivity and specificity depending on the selected cut point (any increase in sensitivity will be accompanied by a decrease in specificity). The area under an ROC curve (AUC) is a measure for the accuracy of a diagnostic test (the larger the area the better; the optimum is 1; a random test would have a ROC curve lying on the diagonal with an area of 0.5. See, e.g.; Egan. Signal Detection Theory and ROC Analysis, Academic Press, New York (1975), incorporated herein by reference.

As used herein, the term “MMSE” refers to a commonly used assessment of cognitive capacity called the Mini-Mental State Examination (see, e.g., Folstein et al., A practical method grading the cognitive state of patients for the clinician, J. Psychiatr Res. vol. 12, no. 3, pp. 189-198 (1975), incorporated herein by reference). During the MMSE, a health professional asks a patient a series of questions designed to test a range of mental skills. The maximum MMSE score is 30 points; a score of 20 to 24 suggests mild dementia; a score of 13 to 20 suggests moderate dementia; and a score of less than 12 indicates severe dementia. One indicator of a subject having Alzheimer's disease is a MMSE score that declines at a rate of approximately two to four points per year.

The term “wild-type” when made in reference to a gene refers to a gene that has the characteristics of a gene isolated from a naturally occurring source. The tem “wild-type” when made in reference to a gene product refers to a gene product that has the characteristics of a gene product isolated from a naturally occurring source. The term “naturally-occurring” as applied to an object refers to the fact that an object can be found in nature. For example, a polypeptide or polynucleotide sequence that is present in an organism (including viruses) that can be isolated from a source in nature and which has not been intentionally modified by man in the laboratory is naturally-occurring. A wild-type gene is frequently that gene which is most frequently observed in a population and is thus arbitrarily designated the “normal” or “wild-type” form of the gene. In contrast, the term “modified” or “mutant” or “variant” when made in reference to a gene or to a gene product refers, respectively, to a gene or to a gene product which displays modifications in sequence and/or functional properties (i.e., altered characteristics) when compared to the wild-type gene or gene product. It is noted that naturally-occurring mutants can be isolated; these are identified by the fact that they have altered characteristics when compared to the wild-type gene or gene product.

Thus, the terms “variant” and “mutant” when used in reference to a nucleotide sequence refer to an nucleic acid sequence that differs by one or more nucleotides from another, usually related nucleotide acid sequence, A “variation” is a difference between two different nucleotide sequences; typically, one sequence is a reference sequence.

As used herein, the term “minor allele frequency” (MAF) refers to the frequency at which the second most common allele occurs in a given population.

As used herein, the term “single nucleotide polymorphism” or “SNP” refers to single nucleotide position in a genomic sequence for which the MAF for the single nucleotide position is 1% or greater.

As used herein, the term “functional single nucleotide polymorphism” or “functional “SNP” refers to a single nucleotide polymorphism that alters the function of a gene or set of genes in a genome, thus causing or ameliorating a disease or providing a readout for a disease, e.g., has a “functional association” with the disease.

As used herein, the term. “tag single nucleotide polymorphism” or “tag SNP” refers to a single nucleotide polymorphism that has a positive statistical association with a disease. A tag single nucleotide polymorphism may be a functional single nucleotide polymorphism or may be associated with the disease by being linked (e.g., in linkage disequilibrium) to a functional single nucleotide polymorphism.

As used herein, “locus” refers to any segment of nucleic acid sequence, e.g., in DNA and defined by chromosomal coordinates in a reference genome known to the art, irrespective of biological function. A locus can contain multiple genes or no genes; a locus can be a single base pair or millions of base pairs; thus, a locus can be a subregion of a nucleic acid, e.g., a gene on a chromosome, a single nucleotide, a CpG island, etc.

As used herein, a “polymorphic locus” is a genomic locus at which two or more alleles have been identified. Thus, the term “polymorphic locus” refers to a genetic locus present in a population that shows variation between members of the population.

As used herein, an “allele” is one of two or more existing genetic variants of a specific polymorphic genomic locus. Thus, the term “allele” refers to different variations in a gene; the variations include but are not limited to variants and mutants, polymorphic loci and single nucleotide polymorphic (SNP) loci, frameshifts, and splice mutations. An allele may occur naturally in a population, or it might arise during the lifetime of any particular individual of the population. When the genetic variation occurs at a SNP locus, the nucleotide variants at the SNP locus are referred to by the term “SNP allele”.

As used herein, a “haplotype” is a unique set of alleles at separate loci that are observed to be inherited as a group (e.g., the alleles segregate together); alleles of a haplotype are often, but are not necessarily, grouped closely together on the same DNA molecule. For instance, in some embodiments, a “haplotype” comprises single nucleotide polymorphisms within a defined region of a chromosome (e.g., within a 50 to 500 kb region of a chromosome (e.g., within 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 kb region). In some embodiments, a “haplotype” comprises a set of single nucleotide polymorphisms that are in linkage disequilibrium, e.g., as measured by an r² value of 0.2 to 0.4 (e.g., 0.20, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, or 0,40), In some embodiments, a “haplotype” comprises a set of single nucleotide polymorphisms that are in a 250-kb region of a chromosome and that are in linkage disequilibrium, e.g., as measured by an r² value of 0.3. Accordingly, a haplotype can be defined by a set of specific alleles at each defined polymorphic locus within a haploblock.

As used herein, a “haploblock” refers to a genomic region that maintains genetic integrity over multiple generations and is recognized by linkage disequilibrium within a population. Haploblocks are defined empirically for a given population of individuals.

As used herein, “linkage disequilibrium” (“LD”) is the non-random association of alleles at two or more loci within a particular population. Linkage disequilibrium is measured as a departure from the null hypothesis of linkage equilibrium, where each allele at one locus associates randomly with each allele at a second locus in a population of individual genomes. Linkage disequilibrium is often measured using an r² value, which is the square of the correlation coefficient between a first indicator variable representing the presence or absence of a particular allele at a first locus and a second indicator representing the presence or absence of a particular allele at a second locus. For example, for two biallelic loci for which the first locus has alleles a and A and the second locus has alleles b and B, and the frequencies for alleles a and A are respectively pₐ and 1−pₐ and the frequencies for alleles b and B are p_{b} and 1−p_{b}, he r² measure of linkage disequilibrium is defined as:

$${r}^{2} ( {p}_{a} , {p}_{b} , {p}_{a b} ) = \frac{{( {p}_{a b} - {p}_{a} {p}_{b} )}^{2}}{{p}_{a} ( 1 - {p}_{a} ) {p}_{b} ( 1 - {p}_{b} )}$$

As used herein, a “genome” is the total genetic information carried by an individual organism or cell, represented by the complete DNA sequences of its chromosomes.

The term “minor allele”, as used herein, refers to the allele that is least frequent in a defined group of individuals when compared with alternative allelic variants at the same genomic position, Minor Allele Frequency (MAF) refers to the frequency of the minor allele in the group.

As used herein, the term “% sequence identity” refers to the percentage of nucleotides or nucleotide analogues in a nucleic acid sequence that is identical with the corresponding nucleotides in a reference sequence after aligning the two sequences and introducing gaps, if necessary, to achieve the maximum percent identity. Hence, in case a nucleic acid according to the technology is longer than a reference sequence, additional nucleotides in the nucleic acid, that do not align with the reference sequence, are not taken into account for determining sequence identity. Methods and computer programs for alignment are well known in the art, including blastn, Align 2, and FASTA.

The term “homology” and “homologous” refers to a degree of identity. There may be partial homology or complete homology. A partially homologous sequence is one that is less than 100% identical to another sequence.

The term “sequence variation” as used herein refers to differences in nucleic acid sequence between two nucleic acids. For example, a wild-type structural gene and a mutant form of this wild-type structural gene may vary in sequence by the presence of single base substitutions and/or deletions or insertions of one or more nucleotides. These two forms of the structural gene are said to vary in sequence from one another. A second mutant form of the structural gene may exist. This second mutant form is said to vary in sequence from both the wild-type gene and the first mutant form of the gene.

The terms “nucleic acid” and “polynucleotide” are used interchangeably herein to describe a polymer of nucleotides (e.g., deoxyribonucleotides and/or ribonucleotides). A nucleic acid can be of any length (e.g., greater than about 2 bases, greater than approximately 10 bases, greater than approximately 100 bases, greater than approximately 500 bases, greater than approximately 1000 bases, and/or up to approximately 10,000 or more bases) and may be natural or synthetic (e.g., produced enzymatically or synthetically). A synthetic nucleic acid can hybridize with naturally occurring nucleic acids in a sequence specific manner analogous to that of two naturally occurring nucleic acids, e.g., can participate in Watson-Crick base pairing interactions. Naturally-occurring nucleotides include guanine, cytosine, adenine, uracil and thymine (G, C, A, U and T, respectively).

Further, as used herein, a “nucleic acid” (e.g., a nucleic acid molecule or sequence) is a deoxyribonucleotide or ribonucleotide polymer including without limitation, cDNA, mRNA, genomic DNA, and synthetic (such as chemically synthesized) DNA or RNA. The nucleic acid can be double-stranded (ds) or single-stranded (ss). Where single-stranded, the nucleic acid can be the sense strand or the antisense strand. Nucleic acids can include natural nucleotides (such as A, T/U, C, and G), and can also include analogs of natural nucleotides, such as labeled nucleotides. Some examples of nucleic acids include the probes disclosed herein. Unless otherwise specified, any reference to a DNA molecule is intended to include the reverse complement of that DNA molecule. DNA molecules, though written to depict only a single strand, encompass both strands of a double-stranded DNA molecule.

The term “oligonucleotide”, as used herein, denotes a single-stranded multimer of nucleotides from approximately 2 to 500 nucleotides (e.g., 2 to 450, 10 to 400, 50 to 350, 100 to 300, or 150 to 200 nucleotides; e.g., approximately 2, 10, 15, 20, 25, 30, 35, 40, 45, 50, 55, 60, 65, 70, 75, 80, 85, 90, 100, 110, 120, 130, 140, 150, 160, 170, 180, 190, 200, 250, 300, 350, 400, 450, or 500 nucleotides). In some embodiments, an oligonucleotide is less than 50 (e.g., under 45, 40, 35, 30, 25, 20, 15, or under 10) nucleotides in length. Oligonucleotides may be 10 to 20, 21 to 30, 31 to 40, 41 to 50, 51 to 60, 61 to 70, 71 to 80, 81 to 100, 101 to 150, or 151 to 200, up to 500 or more nucleotides in length. Oligonucleotides may contain ribonucleotide monomers (e.g., may be oligoribonucleotides) or deoxyribonucleotide monomers. Oligonucleotides may be synthetic or may be made enzymatically.

The term “gene” refers to a nucleic acid (e.g., DNA or RNA) sequence that comprises coding sequences necessary for the production of an RNA, or a polypeptide or its precursor (e.g., proinsulin). A functional polypeptide can be encoded by a full length coding sequence or by any portion of the coding sequence as long as the desired activity or functional properties (e.g., enzymatic activity, ligand binding, signal transduction, etc.) of the polypeptide are retained. The term “portion” when used in reference to a gene refers to fragments of that gene. The fragments may range in size from a few nucleotides to the entire gene sequence minus one nucleotide. Thus, “a nucleotide comprising at least a portion of a gene” may comprise fragments of the gene or the entire gene.

The term “gene” also encompasses the coding regions of a structural gene and includes sequences located adjacent to the coding region on both the 5′ and 3′ ends for a distance of about 1 kb on either end such that the gene corresponds to the length of the full-length mRNA. The sequences which are located 5′ of the coding region and which are present on the mRNA are referred to as 5′ non-translated sequences. The sequences which are located 3′ or downstream of the coding region and which are present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene which are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

In addition to containing introns, genomic forms of a gene may also include sequences located on both the 5′ and 3′ end of the sequences which are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers which control or influence the transcription of the gene. The 3′ flanking region may contain sequences which direct the termination of transcription, posttranscriptional cleavage and polyadenylation.

As used herein, the term “nucleic acid detection assay” refers to any method of determining the nucleotide composition of a nucleic acid of interest. Nucleic acid detection assay include but are not limited to, DNA sequencing methods, probe hybridization methods, allele-specific polymerase chain reaction (PCR), structure specific cleavage assays (see e.g., U.S. Pat. Nos. 5,846,717; 5,985,557; 5,994,069; 6,001,567; 6,090,543; 6,872,816; Lyamichev et al., Nat. Biotech., vol. 17, no. 292 (1999), Hall et al., Proc. Nat'l Acad. Sci. USA, vol. 97, no. 15, p. 8272-8277 (2000), and U.S. Pat. Pub. No. 2009/0253142, each of which is herein incorporated by reference in its entirety for all purposes); enzyme mismatch cleavage methods (e.g., Variagenics, U.S. Pat. Nos. 6,110,684; 5,958,692; and 5,851,770, herein incorporated by reference in their entireties); polymerase chain reaction; branched hybridization methods (e.g., Chiron, U.S. Pat. Nos. 5,849,481; 5,710,264; 5,124,246; and 5,624,802, herein incorporated by reference in their entireties); rolling circle replication (e.g., U.S. Pat. Nos. 6,210,884; 6,183,960; and 6,235,502, herein incorporated by reference in their entireties); NASBA (e.g., U.S. Pat. No. 5,409,818, herein incorporated by reference in its entirety); molecular beacon technology U.S. Pat. No. 6,150,097, herein incorporated by reference in its entirety); E-sensor technology (Motorola, U.S. Pat. Nos. 6,248,229; 6,221,583; 6,013,170 and 6,063,573, herein incorporated by reference in their entireties); cycling probe technology (e.g., U.S. Pat. Nos. 5,403,711; 5,011,769 and 5,660,988, herein incorporated by reference in their entireties); signal amplification methods (e.g., U.S. Pat. Nos. 6,121,001; 6,110,677; 5,914,230; 5,882,867; and 5,792,614, herein incorporated by reference in their entireties); ligase chain reaction (e.g., Barmy, Proc. Natl. Acad. Sci USA vol. 88, no. 1, pp. 189-193 (1991)); and sandwich hybridization methods (e.g., U.S. Pat. No. 5,288,609, herein incorporated by reference in its entirety).

The term “probe,” as used herein, refers to an oligonucleotide. In certain embodiments, a probe may be immobilized on a surface of a substrate, where the substrate can have a variety of configurations, e.g., a sheet, bead, or other structure. In certain embodiments, a probe may be present on a surface of a substantially planar substrate, e.g., in the form of a microarray.

As used herein, the term. “microarray” or “array” refers to a one-dimensional, two-dimensional, or three-dimensional arrangement of addressable regions (“features”), e.g., spatially addressable regions or optically addressable regions, bearing nucleic acid probes, particularly oligonucleotides or synthetic mimetics thereof. In some cases, the addressable regions of the array may not be physically connected to one another, for example, a plurality of beads that are distinguishable by optical or other means may constitute an array. Nucleic acid probes of an array may be adsorbed, physisorbed, chemisorbed, or covalently attached to the arrays at any point or points along the nucleic acid chain and may be attached to the substrate by a linker.

The terms “determining”, “measuring”, “evaluating”, “assessing”, “assaying”, and “analyzing” are used interchangeably herein to refer to any form of measurement and include determining if an element is present or not. These terms include both quantitative and/or qualitative determinations. Assessing may be relative or absolute. “Assessing the presence of” includes determining the amount of something present and/or determining whether it is present or absent.

As used herein, a “diagnostic” test application includes the detection or identification of a disease state or condition of a subject, determining the likelihood that a subject will contract a given disease or condition, determining the likelihood that a subject with a disease or condition will respond to therapy, determining the prognosis of a subject with a disease or condition (or its likely progression or regression), determining the effect of a treatment on a subject with a disease or condition, and/or determining the presence or absence of a pathological marker in a sample. For example, a diagnostic can be used for detecting the presence or likelihood of a subject having cognitive impairment or the likelihood that such a subject will respond favorably to a compound (e.g., a pharmaceutical, e.g., a drug) or other treatment.

The term “marker”, as used herein, refers to a substance (e.g., a nucleic acid or a region of a nucleic acid) or characteristic of a sample or subject that can be detected (e.g., presence can be detected) and/or quantified to provide data, e.g., as input to a machine learning system (i.e., machine learning model) to determine a predictor. In some embodiments, a “marker” is a SNP. In some embodiments, a marker is a functional SNP and in some embodiments a marker is a tag SNP.

As used herein, the term “polygenic risk score” (“PRS”) refers to a value (e.g., a number (e.g., a predictor and/or a classifier) output by a calculation or model using variation at multiple genetic loci and their associated weights as inputs.

The term “corresponding” is a relative term indicating similarity in position, purpose, or structure. For example, a nucleic acid sequence corresponding to a gene promoter indicates that the nucleic acid sequence is similar to the promoter found in an organism; a nucleic acid sequence corresponding to a genome region indicates that the nucleic acid sequence is similar to the sequence found in the genome region found in an organism.

As used herein, the terms “subject” and “patient” refer to any organisms including plants, microorganisms, and animals (e.g., mammals such as dogs, cats, mice, rats, livestock, and humans).

The term “sample” in the present specification and claims is used in its broadest sense. On the one hand it is meant to include a specimen or culture (e.g., microbiological cultures). On the other hand, it is meant to include both biological and environmental samples. A sample may include a specimen of synthetic origin.

As used herein, a “biological sample” refers to a sample of biological tissue or fluid. For instance, a biological sample may be a sample obtained from an animal (including a human); a fluid, solid, or tissue sample; as well as liquid and solid food and feed products and ingredients such as dairy items, vegetables, meat and meat by-products, and waste. Biological samples may be obtained from all of the various families of domestic animals, as well as feral or wild animals, including, but not limited to, such animals as ungulates, bear, fish, lagomorphs; rodents, etc. Examples of biological samples include sections of tissues, blood, blood fractions, plasma, serum, urine, or samples from other peripheral sources or cell cultures, cell colonies, single cells, or a collection of single cells. Furthermore, a biological sample includes pools or mixtures of the above mentioned samples. A biological sample may be provided by removing a sample of cells from a subject, but can also be provided by using a previously isolated sample. For example, a tissue sample can be removed from a subject suspected of having a disease by conventional biopsy techniques. In some embodiments, a blood sample is taken from a subject. A biological sample from a patient means a sample from a subject suspected to be affected by a disease.

Environmental samples include environmental material such as surface matter, soil, water, and industrial samples, as well as samples obtained from food and dairy processing instruments, apparatus, equipment, utensils, disposable and non-disposable items. These examples are not to be construed as limiting the sample types applicable to the present technology.

As used herein, the term “increased risk” refers to an increase in the risk level for a subject to have cognitive impairment relative to a population's known prevalence of cognitive impairment before testing.

### Methods of Cognitive Impairment and Neurodegenerative Pathological Feature Risk Assessment

Although the disclosure herein refers to certain illustrated embodiments, it is to be understood that these embodiments are presented by way of example and not by way of limitation.

In some embodiments, the technology relates to methods for diagnosing and/or identifying a subject who has cognitive impairment and/or who has an increased risk of having cognitive impairment. In some embodiments, the technology relates to methods for identifying a subject who has a neuropathological pathology causative, indicative, and/or associated with a cognitive impairment. In some embodiments, the technology relates to methods for identifying and/or selecting a subject for enrollment in a clinical trial to test a treatment (e.g., a drug or other pharmacological agent) for a cognitive disease. In some embodiments enrollment in clinical trials can be decided based on multiple results of tests characterizing multiple neuropathological pathologies.

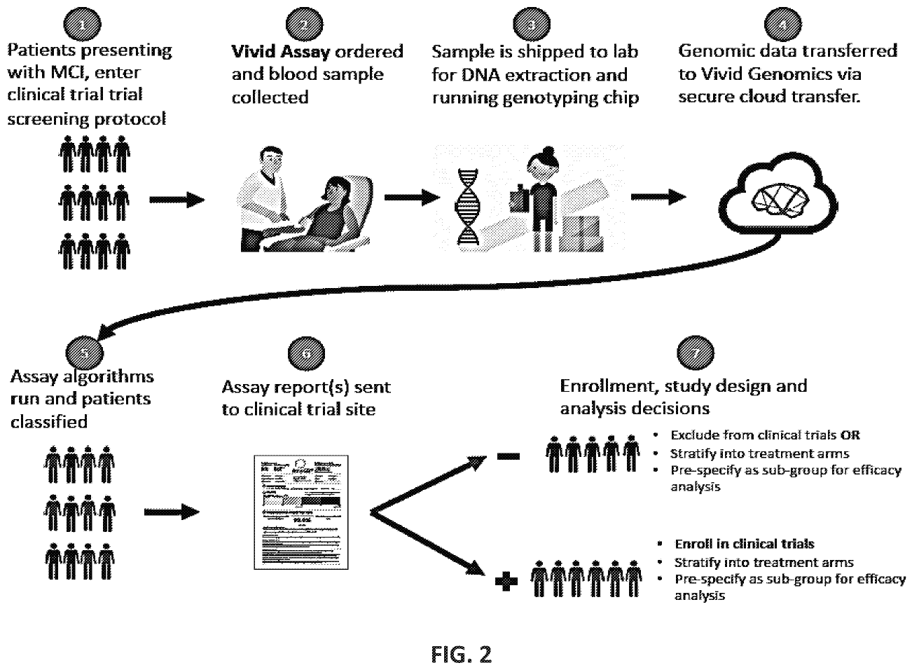

As shown in FIG. 2, in some embodiments, methods comprise providing a subject, e.g., a subject presenting with cognitive impairment (e.g., a mild cognitive impairment). In some embodiments, methods comprise applying a screening protocol to a subject to identify the subject as included in or excluded from a clinical trial, drug treatment, or medical intervention. In some embodiments, methods comprise applying a screening protocol to a subject to identify the subject as enrolled and stratified for a clinical trial, drug treatment, or medical intervention. In some embodiments, methods comprise applying a screening protocol to a subject to identify the subject as enrolled and assigned to sub-groups for analysis in a clinical trial, drug treatment, or medical intervention.

The presence or risk of one or more neurodegenerative pathological features of the cognitive impairment can be predicted or characterized, which can be used to characterize the cognitive impairment. Exemplary features include tau protein, amyloid beta, cerebral amyloid angiopathy (CAA), Lewy bodies, and/or a progression of the cognitive impairment. The characterization of the neurodegenerative pathological features can be based on a panel of markers associated with the neurodegenerative pathological features.

A panel of markers (or markers in linkage disequilibrium with markers in the panel of markers) can be associated with cognitive impairment or a neurodegenerative pathological feature of the cognitive impairment. Different pathological features can have unique panels, although a portion of the markers in the different marker panels may overlap. The status of makers in the panel can be determined, and the determined status can be used, for example by a machine learning system (i.e., a machine learning model), to characterize a presence or risk of the cognitive impairment or one or more neurodegenerative pathological features of the cognitive impairment. The status of the marker can be, for example, a presence or absence of the marker (for example, the presence or absence of a SNP or other genetic variant), or may be some other data point for the marker (such as a specific age of the subject when the marker is an age, or a correlation factor between two or more markers).

A single sample obtained from a patient can be used to characterize two or more neurodegenerative pathological features. For example, a first panel of markers (or markers in linkage disequilibrium with markers in the first panel of markers) may be associated with a first neurodegenerative pathological feature, and a second panel of markers (or markers in linkage disequilibrium with markers in the second panel of markers) may be associated with a second neurodegenerative pathological feature. The different neurodegenerative pathological features have unique marker panels, although there may be some overlap between the marker panels (i.e., a subset of markers may be used in both (or more) marker panels for the two (or more) neurodegenerative pathological features). The status (e.g., presence or absence) of the markers can detected from the same sample obtained from the subject, which allows for characterization of multiple pathological features using a single sample.

In some embodiments, a method for characterizing a plurality of neurodegenerative pathological features of a cognitive impairment in a human subject includes: (a) detecting, in a sample obtained from the subject, the status (e.g., presence or absence) of first markers in a first panel of markers or markers in linkage disequilibrium with markers in the first panel of markers, wherein the first panel of markers is associated with a first neurodegenerative pathological feature of the cognitive impairment; (h) detecting, in the same sample obtained from the subject, the status (e.g., presence or absence) of second markers in a second panel of markers or markers in linkage disequilibrium with markers in the second panel of markers, wherein the second panel of markers is associated with a second neurodegenerative pathological feature of the cognitive impairment; and (c) characterizing a presence or risk the first and second neurodegenerative pathological features of the cognitive impairment in the subject based on the status of the first markers and the status of the second markers. This process can be used to characterize the presence or risk of additional (e.g., 3; 4, 5 or more) neurodegenerative pathological features. In some embodiments, the presence or risk of the different neurodegenerative pathological features are characterized using independently selected machine learning systems (i.e., a machine learning models).

The characterized cognitive impairment (or one or more characterized neurodegenerative pathological features) is used to enroll the subject in a clinical trial. For example, a clinical trial may enroll exclusively or a target subset of subjects that have or do not have the cognitive impairment (or have or do not have one or a combination of neurodegenerative pathological features), or have a risk profile for the cognitive impairment (or risk profile of one or more neurodegenerative pathological features). In some embodiments, the characterization of two or more neurodegenerative pathological features is used to enroll the subject in a clinical trial.

The characterized cognitive impairment (or one or more characterized neurodegenerative pathological features) can also or alternatively be used to determine a course of treatment for the cognitive impairment. In some instances, two or more characterized neurodegenerative pathological features are used to determine a course of treatment for the cognitive impairment.

In some embodiments, methods comprise obtaining a sample from a subject (e.g., providing a sample from a subject and/or receiving a sample from a subject). The technology is not limited in the sample that is obtained from a subject; for instance, in some embodiments, the sample comprises and/or is prepared and/or derived from an organ, a tissue, a cell, and/or a subcellular component (e.g., an organelle) and/or fraction (cell preparation, lysate, etc.) In some embodiments, the sample comprises and/or is prepared and/or derived from a urine, blood, or saliva sample. In some embodiments, the sample comprises and/or is prepared and/or derived from a blood sample (e.g., whole blood, plasma, processed blood, etc.)

In some embodiments, nucleic acid (e.g., DNA or RNA) is isolated from the sample. In some embodiments, a nucleic acid is prepared (e.g., synthesized) using nucleic acid isolated from the sample, e.g., to produce an amplicon, cDNA, or other synthetic nucleic acid representative of one or more nucleic acids present in the sample. In some embodiments, methods comprise determining a genotype from the sample (e.g., providing a genotype of the subject from whom the sample was taken). In some embodiments, genotyping a sample comprises detecting and/or determining the identity of a nucleotide at a position in a human chromosomal location present in a panel of markers and/or detecting and/or determining a nucleotide at a position in a human chromosomal location that is in linkage disequilibrium with a human chromosomal location in the panel of markers, in some embodiments, genotyping a sample comprises detecting and/or determining the identity of a nucleotide at a position in a human chromosomal location provided in Table 1 or Table 2 and/or detecting and/or determining a nucleotide at a position in a human chromosomal location that is in linkage disequilibrium with a human chromosomal location provided in Table 1 or Table 2. Tables 1 and 2 are non-limiting examples of a panel of markers.

In some embodiments, determining a genotype from a sample comprises contacting a genotyping chip (e.g., a microarray) with nucleic acids isolated and/or prepared from a sample to detect a nucleotide at a position in a human chromosomal location provided in a panel of markers or a nucleotide at a position in a human chromosomal location that is in linkage disequilibrium with a human chromosomal location with the panel of markers. Tables 1 and 2 are non-limiting examples of a panel of markers. In some embodiments, determining a genotype from a sample comprises contacting a sample and/or nucleic acids isolated and/or prepared from a sample with a plurality of probes for detecting a nucleotide at a position in a human chromosomal location provided in a panel of markers or a nucleotide at a position in a human chromosomal location that is in linkage disequilibrium with a human chromosomal location provided in the panel of markers. Tables 1 and 2 are non-limiting examples of a panel of markers. In some embodiments, determining a genotype from a sample comprises sequencing nucleic acids isolated and/or prepared from a sample. In some embodiments, sequencing is whole genome sequencing; in some embodiments, sequencing is targeted to a position in a human chromosomal location provided in panel of markers or a nucleotide at a position in a human chromosomal location that is in linkage disequilibrium with a human chromosomal location provided in the panel of markers. Tables 1 and 2 are non-limiting examples of a panel of markers. In some embodiments, genotyping a sample comprises detecting and/or determining a nucleotide at a plurality of human chromosomal locations provided in a panel of markers and/or detecting and/or determining a nucleotide at a plurality of human chromosomal locations that are in linkage disequilibrium with the panel of markers to produce a genetic dataset for the subject. Tables 1 and 2 are non-limiting examples of a panel of markers. In some embodiments, the genetic dataset comprises a collection of nucleotide identities (e.g., A, C, G, or T) associated one-to-one with a collection of human chromosomal locations (e.g., defined by chromosome number and nucleotide position within the chromosome). In some embodiments, clinical and/or therapeutic data are collected from the subject and/or patient. In some embodiments, clinical and/or therapeutic data comprise, e.g., age, biological sex, APOE allele 4 copy number, APOE allele 2 copy number, drug response indicators, symptoms of cognitive ability and/or impairment (e.g., anosmia, memory loss, etc.), score of cognitive ability from a test of cognitive ability (e.g., MMSE score), change with time in a score of cognitive ability from a test of cognitive ability (e.g., change with time of a MMSE score), ethnic and/or racial genotype and/or background, oxidative damage in nucleic acid from the subject, neuroimaging data (e.g., PET, MRI, SPECT), and/or neuropathology (e.g., presence of tau protein, amyloid beta, and/or Lewy bodies; diffuse amyloid in the neocortex and/or neurofibrillary tangles in the medial temporal lobe; and/or loss of grey matter). In some embodiments, the genetic dataset and the clinical and/or therapeutic data are combined to provide a clinico-genetic dataset. In some embodiments the panel of markers comprises DNA structural variants, DNA copy number variants, DNA repeat expansions, DNA STRs, small deletions, large deletions, RNA expression. RNA SNPs, RNA fusions, and DNA methylation.

In some embodiments, determining a genotype from a sample comprises contacting a genotyping chip (e.g., a microarray) with nucleic acids isolated and/or prepared from a sample to detect a nucleotide at a position in a human chromosomal location provided in Table 1 or Table 2 or a nucleotide at a position in a human chromosomal location that is in linkage disequilibrium with a human chromosomal location provided in Table 1 or Table 2. In some embodiments, determining a genotype from a sample comprises contacting a sample and/or nucleic acids isolated and/or prepared from a sample with a plurality of probes for detecting a nucleotide at a position in a human chromosomal location provided in Table 1 or Table 2 or a nucleotide at a position in a human chromosomal location that is in linkage disequilibrium with a human chromosomal location provided in Table 1 or Table 2. In some embodiments, determining a genotype from a sample comprises sequencing nucleic acids isolated and/or prepared from a sample. In some embodiments, sequencing is whole genome sequencing; in some embodiments, sequencing is targeted to a position in a human chromosomal location provided in Table 1 or Table 2 or a nucleotide at a position in a human chromosomal location that is in linkage disequilibrium with a human chromosomal location provided in Table 1 or Table 2. In some embodiments, genotyping a sample comprises detecting and/or determining a nucleotide at a plurality of human chromosomal locations provided in Table 1 or Table 2 and/or detecting and/or determining a nucleotide at a plurality of human chromosomal locations that are in linkage disequilibrium with a human chromosomal location provided in Table 1 or Table 2 to produce a genetic dataset for the subject. In some embodiments, the genetic dataset comprises a collection of nucleotide identities (e.g., A, C, G, or T) associated one-to-one with a collection of human chromosomal locations (e.g., defined by chromosome number and nucleotide position within the chromosome). In some embodiments, clinical and/or therapeutic data are collected from the subject and/or patient. In some embodiments, clinical and/or therapeutic data comprise, e.g., age, biological sex, APOE allele 4 copy number, APOE allele 2 copy number, drug response indicators, symptoms of cognitive ability and/or impairment (e.g., anosmia, memory loss, etc.), score of cognitive ability from a test of cognitive ability (e.g., MMSE score), change with time in a score of cognitive ability from a test of cognitive ability (e.g., change with time of a MMSE score), ethnic and/or racial genotype and/or background, oxidative damage in nucleic acid from the subject, neuroimaging data (e.g., PET, MRI, SPECT), and/or neuropathology (e.g., presence of tau protein, amyloid beta, and/or Lewy bodies; diffuse amyloid in the neocortex and/or neurofibrillary tangles in the medial temporal lobe; and/or loss of grey matter). In some embodiments, the genetic dataset and the clinical and/or therapeutic data are combined to provide a clinico-genetic dataset. In some embodiments the panel of markers comprises DNA structural variants, DNA copy number variants, DNA repeat expansions, DNA STRs, small deletions, large deletions, RNA expression, RNA SNPs, RNA fusions, and DNA methylation.

In some embodiments, a genetic dataset or clinico-genetic dataset is used as input into a patient classifier. In some embodiments, the patient classifier comprises a machine learning model integrating the data in the genetic dataset or clinico-genetic dataset. In some embodiments, the patient classifier comprises a machine learning model integrating the data in the genetic dataset or clinico-genetic dataset and parameters determined from applying the machine learning model to reference samples known to comprise neurodegenerative pathologies and/or known to have been taken from subjects having cognitive impairment. In some embodiments, the machine learning model outputs a classifier and/or a predictor characterizing the subject from whom the genetic dataset or clinico-genetic dataset was produced. Some embodiments comprise producing and/or displaying a report comprising the results (e.g., classifier and/or a predictor) of the machine learning model for the subject. Some embodiments comprise sending a report comprising the results (e.g., classifier and/or a predictor) of the machine learning model for the subject to a clinic, e.g., for use by the clinic in selecting and/or assessing subjects for inclusion and/or exclusion from a clinical trial and/or for selecting and delivering appropriate treatment options for patients.

In some embodiments, the classifier indicates that the subject is included in a clinical trial, drug treatment group, and/or other medical intervention. In some embodiments; the classifier indicates that the subject is excluded from a clinical trial, drug treatment group, and/or other medical intervention. In some embodiments, the predictor indicates that the subject has a neuropathology and/or has increased risk of having a neuropathology. In some embodiments multiple predictors are used to predict different neuropathologies and characterize subjects based on more than one neuropathology prediction. In some embodiments, the predictor indicates that the subject has a cognitive impairment and/or has increased risk of having a cognitive impairment. In some embodiments, the classifier indicates placement of the subject into a risk group and/or is used to indicate the severity and/or stage of cognitive impairment of the subject. In some embodiments, the classifier indicates placement of a subject into a treatment arm of a clinical trial. In some embodiments, the classifier identifies placement of a subject into a sub-group for drug efficacy analysis. In some embodiments more than one classifier for different neuropathologies identifies placement of a subject into a sub-group for drug efficacy analysis or a clinical trial.

In some embodiments, the technology described herein relates generally to the detection or diagnosis of cognitive impairment in a subject. In some embodiments, the technology described herein relates generally to the detection or diagnosis of Alzheimer's disease, dementia, or a prodromal stage of Alzheimer's disease or dementia.

In some embodiments, the technology described herein provides methods, reagents, and kits useful for this purpose. Provided herein are genetic markers that are indicative of and/or diagnostic of cognitive impairment (see, e.g., Table 1 and Table 2 and markers in linkage disequilibrium with a marker in Table 1 or Table 2). In some embodiments, the present technology provides a panel of markers (e.g., genetic markers (e.g., functional SNPs and/or tag SLAPS that indicate the presence of a neuropathology in a patient and/or that indicate that a patient has or has an increased risk of having a cognitive impairment). During the development of the technology provided herein, SNPs and clinical data provided in panel were identified by genotyping reference samples known to comprise a neuropathology and/or known to be taken and/or derived from a subject having a cognitive impairment to produce a genetic and/or clinico-genetic dataset. Then, the experiments applied a machine learning system (i.e., a machine learning model) to the genetic and/or clinico-genetic dataset to produce a classifier and/or predictor indicative of the presence of the neuropathology in the samples and/or indicative of a cognitive impairment in a subject. In some embodiments, genotypes tagging haplotypic variation and known risk loci for neurodegenerative diseases were generated for a reference collection of brain samples with a known pathology (e.g., known to have a neurodegenerative pathology). In some embodiments, genotypes tagging haplotypic variation comprised single nucleotide polymorphisms within a defined region of a chromosome (e.g., within a 50 to 500 kb region of a chromosome (e.g., within 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450. 460, 470, 480, 490, or 500 kb region) and/or single nucleotide polymorphisms that were identified to be in linkage disequilibrium, e.g., as measured by an r² value of 0.2 to 0.4 (e.g., 0.20, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, or 0.40). In some embodiments, genotypes tagging haplotypic variation comprised single nucleotide polymorphisms within approximately 250 kb and having an r² of approximately 0.3. In some embodiments, machine and deep learning methods were used to build unique clinico-genetic models predicting incidence and quantity of the pathological hallmarks of disease in these reference samples. Predictions were carried out in two stages: 1) including only genomic data and 2) including genomic and clinical data (e.g., anosmia, indicators of drug efficiency). In some embodiments, the models were used to extrapolate algorithmic predictions to select candidates for clinical trial enrollment (e.g., candidates having the likely pathology of interest and that, accordingly, would respond to treatment). Previously, machine learning models have been used to produce a similar predictor for Parkinson's disease are described, e.g., in Nalls et al., Lancet Neurology 14: 1002 (2015), incorporated herein by reference. The present technology provides an improvement of these previously described machine learning techniques.

In some embodiments, the present technology provides markers genetic markers (e.g., functional SNPs and/or tag SNPs) and, optionally, clinical and/or therapeutic markers) indicative of cognitive impairment in a subject. In some embodiments, the presence of such markers is indicative of and/or diagnostic of cognitive impairment and/or a neuropathology. In some embodiments, markers are indicative of and/or diagnostic of Alzheimer's disease. In some embodiments, markers are detected from a blood sample. In some embodiments, the present technology provides one or more markers, or a panel of markers, that can be identified from tissue or blood or other sample types. In some embodiments, these markers are present in subjects with current symptoms (e.g., symptoms of cognitive impairment) compared to control subjects (e.g., a subject who does not have a neuropathology and/or who does not exhibit symptoms of cognitive impairment). In some embodiments, the markers modulate levels of one or more proteins expressed from the subject genome in subjects and, accordingly, in some embodiments a protein is a marker as used in the technology.

In some embodiments, a subject to be tested by the methods and reagents described herein exhibits one or more symptoms of cognitive impairment (e.g., Alzheimer's disease and/or dementia). Symptoms of cognitive impairment include, for example: memory loss, confusion, insomnia, paranoia, anxiety, speech problems, apathy, score of cognitive ability from a test of cognitive ability (e.g., MMSE score), change with time (e.g., decline) in a score of cognitive ability from a test of cognitive ability (e.g., change with time (e.g., decline) of a MMSE score), oxidative damage in nucleic acid from the subject, and/or neuropathology (e.g., presence of tau protein, amyloid beta, cerebral amyloid angiopathy (CAA) and/or Lewy bodies; diffuse amyloid in the neocortex and/or neurofibrillary tangles in the medial temporal lobe; and/or loss of grey matter).

In some embodiments, markers (e.g., as provided in Table 1 or Table 2 or markers in linkage disequilibrium with a marker in Table 1 or Table 2) confirm that a subject's symptoms are the result of cognitive impairment. In some embodiments, markers (e.g., as provided in Table 1 or Table 2 or markers in linkage disequilibrium with a marker in Table 1 or Table 2) predict that a subject will develop cognitive impairment at a later time. In some embodiments, markers allow diagnosis of cognitive impairment in a subject not actively experiencing and/or exhibiting symptoms or unable to communicate such symptoms. In some embodiments, markers differentiate between a subject experiencing symptoms caused by cognitive impairment and those caused by another cause, e.g., stress or other disease.

The present technology relates to the use of a panel of markers (for example as shown for example in Table 1 or Table 2) or markers in linkage disequilibrium with the panel of markers (for example the panel of marker in Table 1 or Table 2) and/or the use thereof in detecting, characterizing, identifying, and/or diagnosing cognitive impairment in a subject. Experiments were conducted during development of embodiments of the present technology to identify markers that are indicative and/or diagnostic of cognitive impairment and/or neuropathologics and to develop a machine learning system (i.e., a machine learning model) for producing a classifier or predictor of cognitive impairment. In some embodiments, markers as provided in Table 1 or Table 2 or markers in linkage disequilibrium with a marker in Table 1 or Table 2 find use in diagnosis and/or characterization of cognitive impairment. In some embodiments, markers of markers as provided in Table 1 or Table 2 or markers in linkage disequilibrium with a marker in Table 1 or Table 2 are indicative of cognitive impairment. In some embodiments, markers of Table 1 or a marker in linkage disequilibrium with a marker in Table 1 finds use in classifying cognitive disease progression in a subject. In some embodiments markers not present in Table 1 or Table 2 or a marker in linkage disequilibrium with a marker not present in Table 1 or Table 2 are used in classifying cognitive disease progression in a subject. In some embodiments, disease progression classes are stratified by speed of decline in cognitive ability with time change in score of a test of cognitive ability (e.g., MMSE or CDR-SB) with time). In some embodiments, disease progression classes are stratified by cognitive ability as assessed by a neuropsychological test of cognitive ability (e.g., MMSE or CDR-SB). In some embodiments, disease progression classes are stratified by different patterns of change in score of a test of cognitive ability (e.g., MMSE or CDR-SB) as a function of time. In some embodiments, markers of Table 2 or a marker in linkage disequilibrium with a marker in Table 2 finds use in indicating the presence of a neurodegenerative pathology in a subject (e.g., indicative of tau protein, amyloid beta, cerebral amyloid angiopathy (CAA) and/or Lewy bodies in the subject).

In some embodiments, a panel of markers for characterization and/or diagnosis of cognitive impairment comprises markers as provided in Table 1 or Table 2 or markers in linkage disequilibrium with a marker in Table 1 or Table 2. In some embodiments, the present technology provides a panel of markers comprising a plurality (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 or more) markers as provided in Table 1 or Table 2 or markers in linkage disequilibrium with a marker in Table 1 or Table 2.

In some embodiments, the present technology provides a panel of reagents for detecting SNPs (e.g., a functional SNP or a tag SNP) from one or more loci as provided a panel of markers (e.g. Table 1 or Table 2 or markers in linkage disequilibrium with a marker in Table 1 or Table 2). In some embodiments, a panel comprises one or more reagents for detecting SNPs (e.g., a functional SNP or a tag SNP) from one or more loci as provided in a panel of markers (e.g. Table 1 or Table 2 or markers in linkage disequilibrium with a marker in Table 1 or Table 2) and one or more additional genes. In some embodiments of the present technology, the presence in a sample of one or more SNPs (e.g., a functional SNP or a tag SNP) from one or more loci as provided a panel of or markers in linkage disequilibrium with a the panel of markers is/are used to diagnose or suggest a risk of cognitive impairment in a human from which the sample was taken. In some embodiments, the presence in a sample of one or more SNPs a functional SNP or a tag SNP) from one or more loci as provided in a panel of markers or markers in linkage disequilibrium with a marker allows a treating physician to take any number of courses of action, including, but not limited to, further diagnostic assessment, selection of appropriate treatment (e.g., pharmacological, nutritional, counseling, and the like), increased or decreased monitoring, etc.

In some embodiments, the present technology provides a method for detecting or assessing the risk of a subject developing a cognitive impairment or one or more neurodegenerative pathological features associated with a cognitive impairment. In some embodiments, the present technology provides a method for diagnosing a cognitive impairment in a subject. In some embodiments, the markers provided herein are used in conjunction with other evidence of cognitive impairment (e.g., symptoms, risk factors, etc.) in making a diagnosis. In some embodiments, the markers provided herein are used in the absence of other evidence of cognitive impairment (e.g., symptoms) in making a diagnosis.

In some embodiments, the present technology provides methods for characterizing a genome and/or a genetic profile of a subject by detecting the presence in a sample from the subject (e.g., a blood sample) of one or more SNPs (e.g., a functional SNP or a tag SNP) from one or more loci as provided in a panel of markers (for example as provide in Table 1 or Table 2) or markers in linkage disequilibrium with a marker in the panel of markers (for example as provide in Table 1 or Table 2). In some embodiments, the panel comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71.72, 73, 74, 75, 76, 77, 78, 79, 80, 81, 82, 83, 84, 85, 86, 87, 88, 89, 90, 91, 92, 93, 94, 95, 96, 97, 98, 99, or 100 or more markers (e.g., SNPs (e.g., functional SNPs and/or tag SNPs). In some embodiments, the present technology provides methods comprising the step of exposing a sample to nucleic acid probes complementary to nucleic acids comprising functional SNPs and/or tag SNPs of a panel of SNPs selected from the markers in a panel of markers (for example as provided in Table 1 or Table 2) or markers in linkage disequilibrium with a marker in the panel of markers (for example, as provided in Table 1 or Table 2). In some embodiments, the methods employ a nucleic acid detection technique (e.g., microarray analysis, nucleic acid amplification, quantitative nucleic acid amplification, digital PCR, and hybridization analysis (e.g., comprising use of oligonucleotide probes)). In some embodiments, the methods employ a nucleic acid sequencing technique. In some embodiments, methods employ a technique that is, e.g., dynamic allele-specific hybridization, molecular beacon, SNP microarray, restriction fragment length polymorphism, a flap endonuclease method, primer extension, 5′-nuclease method, oligonucleotide ligation assay, single-strand conformation polymorphism, temperature gradient gel electrophoresis, denaturing HPLC, high-resolution melting curve, nucleic acid sequencing, and/or a surveyor nuclease assay.

In some embodiments, the present technology provides a panel of markers for the detection, characterization, and/or diagnosis of a variety of diseases and/or conditions (e.g., psychiatric conditions, mental disease, genetic conditions, physical diseases, etc.), one of which is cognitive impairment. In some embodiments, a panel comprises multiple markers from the markers in a panel of markers (for example as provided in Table 1 or Table 2) or markers in linkage disequilibrium with a marker in the panel of markers (for example as provided in Table 1 or Table 2) in addition to markers for other diseases or conditions (e.g., depression, anxiety, etc.). In particular embodiments, testing a subject (e.g., testing a sample from a subject (e.g., testing a blood sample from a subject)) for such a panel allows diagnosis of cognitive impairment in addition to other diseases, conditions, or disorders. In some embodiments, all the markers on the panel are provided for a diagnostic or other medical purpose.

It is contemplated that a test sample (e.g., containing isolated and/or purified nucleic acid (e.g., genomic DNA, amplicon produced from genomic DNA, etc.), containing test reagents, etc.) is prepared from a biological sample (e.g., saliva, blood, etc.) from a subject (e.g., with cognitive impairment or in need of testing for cognitive impairment), and the test samples are applied to the panel. In some embodiments, the differential hybridization of a patient sample relative to a control sample provides a genetic and/or genomic profile for cognitive impairment and/or a genetic dataset for input into a machine learning algorithm to produce a classifier. In some embodiments, a genetic and/or genomic profile and/or a classifier from a test sample is compared with a genetic and/or genomic profile and/or a classifier from a prior sample from the same patient to monitor changes over time. In some embodiments, a genetic and/or genomic profile and/or a classifier from a test sample is compared with a sample from the patient under a treatment regimen (e.g., pharmaceutical therapy) to test or monitor the effect of the therapy. In some embodiments, a genetic and/or genomic profile and/or a classifier from a test sample is compared to a genetic and/or genomic profile and/or a classifier from a negative control sample (e.g., a subject known not to have cognitive impairment). In some embodiments, a genetic and/or genomic profile and/or a classifier from a test sample are compared to a predetermined threshold level previously identified and/or known (e.g., based on population averages for patients with similar age, biological sex, metabolism, etc.) as “normal” for individuals without cognitive impairment.

In some embodiments, provided herein are nucleic acid-based diagnostic methods that either directly or indirectly detect the markers described herein. The present technology also provides compositions, reagents, and kits for such diagnostic purposes. The diagnostic methods described herein may be qualitative (e.g., presence or absence of cognitive impairment) or quantitative (e.g., classification and/or measurement of cognitive impairment).

In some embodiments, markers are detected at the nucleic acid (e.g., DNA) level. For example, the presence of a SNP in a sample is determined. In some embodiments, the SNP is characterized as: 1) absent, 2) present and heterozygous, or 3) present and homozygous. Marker nucleic acid (e.g., SNPs) may be detected/quantified using a variety of nucleic acid techniques known to those of ordinary skill in the art, including but not limited to nucleic acid sequencing, nucleic acid hybridization, and nucleic acid amplification.

In some embodiments, a microarray is used to detect nucleic acid markers from a panel of nucleic acid markers (e.g., as provided in Table 1 or Table 2) or markers in linkage disequilibrium with a nucleic acid marker from the panel of nucleic acid markers (e.g., a marker in disequilibrium with a nucleic acid marker in Table 1 or Table 2). Different kinds of biological assays are called microarrays including, but not limited to: DNA microarrays (e.g., oligonucleotide microarrays); protein microarrays; tissue microarrays; transfection or cell microarrays; chemical compound microarrays; and, antibody microarrays. A DNA microarray, commonly known as gene chip, DNA chip, or biochip, is typically a collection of microscopic DNA spots attached to a solid surface (e.g., glass, plastic or silicon chip) forming an array for the purpose of detecting the presence or absence of thousands of markers (e.g., SNPs) simultaneously. The affixed DNA segments are known as probes, thousands of which can be used in a single DNA microarray. Microarrays can be used to identify disease markers by comparing markers in disease and normal cells. Microarrays can be fabricated using a variety of technologies, including but not limiting: printing with fine-pointed pins onto glass slides; photolithography using pre-made masks; photolithography using dynamic micromirror devices; ink jet printing; or, electrochemistry on microelectrode arrays.

In some embodiments the nucleic acid markers comprise DNA structural variants, DNA copy number variants, DNA repeat expansions, DNA STRs, small deletions, large deletions, RNA expression, RNA SNPs, RNA fusions, and DNA methylation.

In some embodiments, the technology comprises use of a probe hybridization method, e.g., using immobilize nucleic acid from a sample (e.g., Southern blotting) or using a solution hybridization method (e.g., FISH). DNA extracted from a sample is fragmented, electrophoretically separated on a matrix gel, and transferred to a membrane filter. The filter hound DNA is subject to hybridization with a labeled probe complementary to the sequence of interest. Hybridized probe bound to the filter is detected.

In some embodiments, genomic DNA is amplified prior to or simultaneous with detection. Illustrative non-limiting examples of nucleic acid amplification techniques include, but are not limited to, polymerase chain reaction (PCR), transcription-mediated amplification (TMA), ligase chain reaction (LCR), strand displacement amplification (SDA), and nucleic acid sequence based amplification (NASBA).

The polymerase chain reaction (U.S. Pat. Nos. 4,683,195; 4,683,202; U.S. Pat. Nos. 4,800,159; and 4,965,188, each of which is herein incorporated by reference in its entirety), commonly referred to as PCR, uses multiple cycles of denaturation, annealing of primer pairs to opposite strands, and primer extension to exponentially increase copy numbers of a target nucleic acid sequence. In some embodiments, PCR is digital PCR, see, e.g., Vogelstein, B., & Kinzler, K. W., Digital PCR. Proc. Natl. Acad. Sci. USA vol. 96, pp. 9236-9241 (1999); herein incorporated by reference in its entirety. For other various permutations of PCR see, e.g., U.S. Pat. Nos. 4,683,195; 4,683,202; and 4,800,159; and Mullis et al., Meth. Enzymol., vol., 155, p. 335-350 (1987), each of which is herein incorporated by reference in its entirety.

Transcription mediated amplification (U.S. Pat. Nos. 5,480,784 and 5,399,491, each of which is herein incorporated by reference in its entirety), commonly referred to as TMA, synthesizes multiple copies of a target nucleic acid sequence autocatalytically under conditions of substantially constant temperature, ionic strength, and pH in which multiple RNA copies of the target sequence autocatalytically generate additional copies. See, e.g., U.S. Pat. Nos. 5,399,491 and 5,824,518, each of which is herein incorporated by reference in its entirety. In a variation described in U.S. Publ, No. 2006/0046265 (herein incorporated by reference in its entirety), TMA optionally incorporates the use of blocking moieties, terminating moieties, and other modifying moieties to improve TMA process sensitivity and accuracy.

The ligase chain reaction (Weiss, Hot Prospect far New Gene Amplifier, Science, vol. 254, pp. 1292-1293 (1991), herein incorporated by reference in its entirety), commonly referred to as LCR, uses two sets of complementary DNA oligonucleotides that hybridize to adjacent regions of the target nucleic acid. The DNA oligonucleotides are covalently linked by a DNA ligase in repeated cycles of thermal denaturation, hybridization and ligation to produce a detectable double-stranded ligated oligonucleotide product.

Strand displacement amplification (Walker, G. et al., Proc. Natl. Acad, Sci. USA vol. 89, pp. 392-396 (1992); U.S. Pat. Nos. 5,270,184 and 5,455,166, each of which is herein incorporated by reference in its entirety), commonly referred to as SDA, uses cycles of annealing pairs of primer sequences to opposite strands of a target sequence, primer extension in the presence of a dNTPαS to produce a duplex hemiphosphorothioated primer extension product, endonuclease-mediated nicking of a hemimodified restriction endonuclease recognition site, and polymerase-mediated primer extension from the 3′ end of the nick to displace an existing strand and produce a strand for the next round of primer annealing, nicking and strand displacement, resulting in geometric amplification of product. Thermophilic SDA (tSDA) uses thermophilic endonucleases and polymerases at higher temperatures in essentially the same method (see, e.g., EP Pat. Pub. 0684315, incorporated herein by reference).

Other amplification methods include, for example: nucleic acid sequence based amplification (U.S. Pat. No. 5,130,238, herein incorporated by reference in its entirety), commonly referred to as NASBA; one that uses an RNA replicase to amplify the probe molecule itself (Lizardi et al, BioTechnol. vol. 6, p. 1197 (1988), herein incorporated by reference in its entirety), commonly referred to as Qβ replicase; a transcription based amplification method (Kwoh et al., Proc. Natl. Acad. Sci, USA vol. 86, p. 1173 (1989)); and, self-sustained sequence replication (Guatelli et al., Proc. Natl. Acad. Sci. USA, vol. 87, p. 1874 (1990), each of which is herein incorporated by reference in its entirety), For further discussion of known amplification methods see Persing, David H., “In Vitro Nucleic Acid Amplification Techniques” in Diagnostic Medical Microbiology: Principles and Applications (Persing et al., Eds.), pp. 51-87 (American Society for Microbiology, Washington, DC (1993)).

Non-amplified or amplified nucleic acids can be detected by any conventional means. For example, in some embodiments, nucleic acids are detected by hybridization with a detectably labeled probe and measurement of the resulting hybrids. Illustrative non-limiting examples of detection methods are described below.

One illustrative detection method, the Hybridization Protection Assay (HPA) involves hybridizing a chemiluminescent oligonucleotide probe (e.g., an acridinium ester-labeled (AE) probe) to the target sequence, selectively hydrolyzing the chemiluminescent label present on unhybridized probe, and measuring the chemiluminescence produced from the remaining probe in a luminometer. See, e.g., U.S. Pat. No. 5,283,174 and Norman C. Nelson et al., Nonisotopic. Probing, Blotting, and Sequencing, ch. 17 (Larry J. Kricka ed., 2d ed. 1995), each of which is herein incorporated by reference in its entirety.

Another illustrative detection method provides for quantitative evaluation of the amplification process in real-time. Evaluation of an amplification process in “real-time” involves determining the amount of amplicon in the reaction mixture either continuously or periodically during the amplification reaction, and using the determined values to calculate the presence and/or amount of target sequence initially present in the sample. A variety of methods for determining the presence and/or amount of initial target sequence present in a sample based on real-time amplification are well known in the art. These include methods disclosed in U.S. Pat. Nos. 6,303,305 and 6,541,205, each of which is herein incorporated by reference in its entirety. Another method for determining the presence and/or quantity of target sequence initially present in a sample, but which is not based on a real-time amplification, is disclosed in U.S. Pat. No. 5,710,029, herein incorporated by reference in its entirety.

Amplification products may be detected in real-time through the use of various self-hybridizing probes, most of which have a stem-loop structure. Such self-hybridizing probes are labeled so that they emit differently detectable signals, depending on whether the probes are in a self-hybridized state or an altered state through hybridization to a target sequence. By way of non-limiting example, “molecular torches” are a type of self-hybridizing probe that includes distinct regions of self-complementarity (referred to as “the target binding domain” and “the target closing domain”) which are connected by a joining region (e.g., non-nucleotide linker) and which hybridize to each other under predetermined hybridization assay conditions. In a preferred embodiment, molecular torches contain single-stranded base regions in the target binding domain that are from 1 to about 20 bases in length and are accessible for hybridization to a target sequence present in an amplification reaction under strand displacement conditions. Under strand displacement conditions, hybridization of the two complementary regions, which may be fully or partially complementary, of the molecular torch is favored, except in the presence of the target sequence, which will bind to the single-stranded region present in the target binding domain and displace all or a portion of the target closing domain. The target binding domain and the target closing domain of a molecular torch include a detectable label or a pair of interacting labels (e.g., luminescent/quencher) positioned so that a different signal is produced when the molecular torch is self-hybridized than when the molecular torch is hybridized to the target sequence, thereby permitting detection of probe:target duplexes in a test sample in the presence of unhybridized molecular torches. Molecular torches and a variety of types of interacting label pairs are disclosed in U.S. Pat. No. 6,534,274, herein incorporated by reference in its entirety.

Another example of a detection probe having self-complementarity is a “molecular beacon.” Molecular beacons include nucleic acid molecules having a target complementary sequence, an affinity pair (or nucleic acid arms) holding the probe in a closed conformation in the absence of a target sequence present in an amplification reaction, and a label pair that interacts when the probe is in a closed conformation. Hybridization of the target sequence and the target complementary sequence separates the members of the affinity pair, thereby shifting the probe to an open conformation. The shift to the open conformation is detectable due to reduced interaction of the label pair, which may be, for example, a fluorophore and a quencher (e.g., DABCYL and EDANS). Molecular beacons are disclosed in U.S. Pat. Nos. 5,925,517 and 6,150,097, herein incorporated by reference in its entirety.

Other self-hybridizing probes are well known to those of ordinary skill in the art. By way of non-limiting example, probe binding pairs having interacting labels, such as those disclosed in U.S. Pat. No. 5,928,862 (herein incorporated by reference in its entirety) might be adapted for use in the present technology. Additional detection systems include “molecular switches,” as disclosed in U.S. Pub. No. 2005/0042638, herein incorporated by reference in its entirety. Other probes, such as those comprising intercalating dyes and/or fluorochromes, are also useful for detection of amplification products in the present technology. See, e.g., U.S. Pat. No. 5,814,447 (herein incorporated by reference in its entirety).

In some embodiments, quantitative PCR (qPCR) is utilized, e.g., using SYBR Green dye on an Applied Biosystems 7300 Real Time PCR system essentially as described (Chinnaiyan et al., Cancer Res. vol. 65, p. 3328 (2005); Rubin et al., Cancer Res. vol. 64, p. 3814 (2004); herein incorporated by reference in its entirety).

In some embodiments, nucleic acid from a sample is sequenced (e.g., in order to detect markers). Nucleic acid molecules may be sequence analyzed by any number of techniques. The analysis may identify the sequence of all or a part of a nucleic acid. Illustrative non-limiting examples of nucleic acid sequencing techniques include, but are not limited to, chain terminator (Sanger) sequencing and dye terminator sequencing, as well as “next generation” sequencing techniques. Those of ordinary skill in the art will recognize that because RNA is less stable in the cell and more prone to nuclease attack, experimentally RNA is usually, although not necessarily, reverse transcribed to DNA before sequencing.

A number of DNA sequencing techniques are known in the art, including fluorescence-based sequencing methodologies (See, e.g., Birren et al., Genome Analysis: Analyzing DNA, 1, Cold Spring Harbor, N.Y.; herein incorporated by reference in its entirety). In some embodiments, automated sequencing techniques understood in that art are utilized. In some embodiments, the systems, devices, and methods employ parallel sequencing of partitioned amplicons (PCT Pub. No: WO2006/084132, herein incorporated by reference in its entirety). In some embodiments, DNA sequencing is achieved by parallel oligonucleotide extension (See, e.g., U.S. Pat. Nos. 5,750,341 and 6,306,597, both of which are herein incorporated by reference in their entireties). Additional examples of sequencing techniques include the Church polony technology (Mitra et al., Analytical Biochemistry vol. 320, pp. 55-65 (2003); Shendure et al., Science vol. 309, pp. 1728-1732 (2005); U.S. Pat. Nos. 6,432,360; 6,485,944; 6,511,803; herein incorporated by reference in their entireties), the 454 picotiter pyrosequencing technology (Margulies et al., Nature vol, 437, pp. 376-380 (2005); U.S. Pat. Pub. No. 2005/0130173; herein incorporated by reference in their entireties), the Solexa single base addition technology (Bennett et al., Pharmacogenomics, vol. 6, pp. 373-382 (2005); U.S. Pat. Nos. 6,787,308; and 6,833,246; herein incorporated by reference in their entireties), the Lynx massively parallel signature sequencing technology (Brenner et al., Nat. Biotechnol. Vol. 18, pp. 630-634 (2000); U.S. Pat. Nos. 5,695,934; 5,714,330; herein incorporated by reference in their entireties), and the Adessi PCR colony technology (Adessi et al., Nucleic Acid Res. vol. 28, p. E87 (2000); PCT Publication No. WO 00/018957; herein incorporated by reference in its entirety).

A set of methods referred to as “next-generation sequencing” techniques have emerged as alternatives to Sanger and dye-terminator sequencing methods (Voelkerding et al., Clinical Chem., vol. 55, pp. 641-658 (2009); MacLean et al., Nature Rev. Microbiol., 7, pp. 287-296 (2009); each herein incorporated by reference in their entirety). Next-generation sequencing (NGS) methods share the common feature of massively parallel, high-throughput strategies, with the goal of lower costs in comparison to older sequencing methods. NGS methods can be broadly divided into those that require template amplification and those that do not. Amplification-requiring methods include pyrosequencing commercialized by Roche as the 454 technology platforms (e.g., GS 20 and GS FLX), the Solexa platform commercialized by Illumina, and the Supported Oligonucleotide Ligation and Detection (SOLiD) platform commercialized by Applied Biosystems. Non-amplification approaches, also known as single-molecule sequencing, are exemplified by the HeliScope platform commercialized by Helicos BioSciences, Pacific Biosciences (PAC BIO RS II), nanopore sequencing, and other platforms commercialized.

In some embodiments, methods comprise isolating nucleic acid DNA) from a biological sample. Methods may comprise steps of homogenizing a sample in a suitable buffer, removal of contaminants and/or assay inhibitors adding a target capture reagent (e.g., a magnetic bead to which is linked an oligonucleotide complementary to the target), incubated under conditions that promote the association (e.g., by hybridization) of the target with the capture reagent to produce a target:capture reagent complex, incubating the target:capture complex under target-release conditions. In some embodiments, multiple marker targets are isolated in each round of isolation by adding multiple target capture reagents (e.g., specific to the desired markers) to the solution. For example, multiple target capture reagents, each comprising an oligonucleotide specific for a different marker target can be added to the sample for isolation of multiple targets. It is contemplated that the methods encompass multiple experimental designs that vary both in the number of capture steps and in the number of targets captured in each capture step. In some embodiments, capture reagents are molecules, moieties, substances, or compositions that preferentially (e.g., specifically and selectively) interact with a particular marker sought to be isolated, purified, detected, and/or quantified. Any capture reagent having desired binding affinity and/or specificity to the analyte target can be used in the present technology. For example, the capture reagent can be a macromolecule such as a peptide, a protein (e.g., an antibody or receptor), an oligonucleotide, a nucleic acid, (e.g., nucleic acids capable of hybridizing with the target nucleic acids), vitamins, oligosaccharides, carbohydrates, lipids, or small molecules, or a complex thereof. As illustrative and non-limiting examples, an avidin target capture reagent may be used to isolate and purify targets comprising a biotin moiety, an antibody may be used to isolate and purify targets comprising the appropriate antigen or epitope, and an oligonucleotide may be used to isolate and purify a complementary oligonucleotide.

Any nucleic acids, including single-stranded and double-stranded nucleic acids that are capable of binding, or specifically binding, to the target can be used as the capture reagent. Examples of such nucleic acids include DNA, RNA, aptamers, peptide nucleic acids, and other modifications to the sugar, phosphate, or nucleoside base. Thus, there are many strategies for capturing a target and accordingly many types of capture reagents are known to those in the art.

In addition, target capture reagents comprise a functionality to localize, concentrate, aggregate, etc. the capture reagent and thus provide a way to isolate and purify the target marker when captured (e.g., bound, hybridized, etc.) to the capture reagent (e.g., when a target:capture reagent complex is formed). For example, in some embodiments the portion of the target capture reagent that interacts with the target (e.g., the oligonucleotide) is linked to a solid support (e.g., a bead, surface, resin, column, and the like) that allows manipulation by the user on a macroscopic scale. Often, the solid support allows the use of a mechanical means to isolate and purify the target:capture reagent complex from a heterogeneous solution. For example, when linked to a bead, separation is achieved by removing the bead from the heterogeneous solution, e.g., by physical movement. In embodiments in which the bead is magnetic or paramagnetic, a magnetic field is used to achieve physical separation of the capture reagent (and thus the target) from the heterogeneous solution. Magnetic beads used to isolate targets are described in the art.

In some embodiments, a computer-based analysis program is used to translate the raw data generated by the detection assay (e.g., the presence, absence, or heterozygous/homozygous state of a SNP) into data of predictive value for a clinician (e.g., a risk score, a qualitative description, etc.). In some embodiments, data analysis produces a cognitive impairment risk or likelihood score. In some embodiments, data analysis produces a cognitive impairment diagnosis. In some embodiments, computer analysis combines the data from numerous markers into a single score or value that is predictive and/or diagnostic for cognitive impairment, e.g., using a machine learning system (i.e., a machine learning model).

In some embodiments, a clinician accesses the data and/or analysis thereof using any suitable means. Thus, in some preferred embodiments, the present technology provides the fluffier benefit that the clinician, who is not likely to be trained in genetics or molecular biology, need not understand the raw data. The data is presented directly to the clinician in its most useful form. The clinician is then able to immediately utilize the information in order to optimize the care of the subject.

The present technology contemplates any method capable of receiving, processing, and transmitting the information to and from laboratories conducting the assays, information providers, medical personnel, and subjects. For example, in some embodiments of the present technology, a sample (e.g., a biopsy or a blood, serum, or saliva sample) is obtained from a subject and submitted to a profiling service (e.g., a clinical lab at a medical facility, a third-party testing service, a genomic profiling business, etc.), located in any part of the world (e.g., in a country different than the country where the subject resides or where the information is ultimately used) to generate raw data. Where the sample comprises a tissue or other biological sample, the subject may visit a medical center to have the sample obtained and sent to the profiling center, or subjects may collect the sample themselves (e.g., a blood or saliva sample, a urine sample, etc.) and directly send it to a profiling center. Where the sample also comprises previously determined biological information, the information may be directly sent to the profiling service by the subject (e.g., an information card containing the information may be scanned by a computer and the data transmitted to a computer of the profiling center using an electronic communication systems). Once received by the profiling service, the sample is processed and a profile is produced (e.g., marker data), specific for the diagnostic or prognostic information desired for the subject.

In some embodiments, profile data is prepared in a format suitable for interpretation by a treating clinician and/or the test subject. For example, rather than providing raw expression data, the prepared format may represent a diagnosis or risk assessment (e.g., likelihood of subject having cognitive impairment) for the subject. Recommendations for particular treatment options and/or placement into particular clinical trial groups may also be provided. The data may be displayed to the clinician by any suitable method. For example, in some embodiments, the profiling service generates a report that can be printed for the clinician (e.g., at the point of care) or displayed to the clinician on a computer monitor.

In some embodiments, a report is generated (e.g., by a clinician, by a testing center, by a computer or other automated analysis system, etc.). A report may contain test results, diagnoses (e.g., cognitive impairment, high likelihood of cognitive impairment, severe cognitive impairment, etc. and/or treatment recommendations (e.g., psychoanalysis, psychotherapy, pharmaceutical treatment, observation, etc.) or placement into a clinical trial group.

In some embodiments, the information is first analyzed at the point of care or at a regional facility. The raw data is then sent to a central processing facility for further analysis and/or to convert the raw data to information useful for a clinician or patient. The central processing facility provides the advantage of privacy (all data is stored in a central facility with uniform security protocols), speed, and uniformity of data analysis. The central processing facility can then control the fate of the data following treatment of the subject. For example, using an electronic communication system, the central facility can provide data to the clinician, the subject, or researchers.

In some embodiments, the subject is able to directly access the data using an electronic communication system. The subject may choose further intervention, treatment, and/or counseling based on the results. In some embodiments, the data is used for research use. For example, the data may be used to further optimize the inclusion or elimination of markers as more or less useful indicators of cognitive impairment (e.g., in a particular population (e.g., children, adolescents, adults, males, females, etc.)

Compositions for use in the diagnostic methods of the present technology include, but are not limited to, probes and amplification oligonucleotides and arrays. Systems and kits are provided that are useful, necessary, and/or sufficient for detecting the presence of one or more markers.

Any of these compositions, alone or in combination with other compositions of the present technology, may be provided in the form of a kit or reagent mixture. For example, in some embodiments, primer pairs and labeled probes are provided in a kit for the amplification and detection of a panel of markers (for example a panel of markers selected from those provided in Table 1 or Table 2) or markers in linkage disequilibrium with a marker in the panel of markers (for example the panel of markers In Table 1 or Table 2). In some embodiments, kits comprise an array for the detection of a panel of markers (for example) or markers in linkage disequilibrium with a marker in the panel of markers (for example those in Table 1 or Table 2). In some embodiments, kits comprise primer pairs and an array for the amplification and detection of a panel of markers (for example, a panel of markers selected from those provided in Table 1 or Table 2) or markers in linkage disequilibrium with a marker in the panel of markers (for example, the panel of markers in Table 1 or Table 2). Kits may include any and all components necessary or sufficient for assays including, but not limited to, detection reagents, amplification reagents, buffers, control reagents (e.g., tissue samples, positive and negative control sample, etc.), solid supports, labels, written and/or pictorial instructions and product information, inhibitors, labeling and/or detection reagents, package environmental controls (e.g., ice, desiccants, etc.), and the like. In some embodiments, kits provide a sub-set of the required components, wherein it is expected that the user will supply the remaining components. In some embodiments, the kits comprise two or more separate containers wherein each container houses a subset of the components to be delivered.

In some embodiments, the present technology provides therapies for diseases characterized by the presence of one or more markers identified using the methods of the present technology and/or the identity of the nucleotide present at one or more marker positons (for example, as provided in Table 1 or Table 2) or markers in linkage disequilibrium with a marker at one or more marker positons (for example as provided in Table 1 or Table 2). In particular, the present technology provides methods and compositions for monitoring the effects of a candidate therapy and for selecting therapies for patients (e.g., for selecting subjects for enrollment in a clinical trial).

In some embodiments, methods of treating cognitive impairment are provided (e.g., following marker identification of a subject as suffering from cognitive impairment). Suitable treatments include psychotherapy, medication, and surgery.

In some embodiments, systems and devices are provided for implementing the diagnostic methods described herein (e.g., data analysis, communication, result reporting, etc.). In some embodiments, a software or hardware component receives the results of multiple assays, factors, and/or markers and determines a single value result to report to a user that indicates a conclusion (e.g., high risk of cognitive impairment, low risk of cognitive impairment, cognitive impairment diagnosis, etc.). Related embodiments calculate a risk factor based on a mathematical combination (e.g., a weighted combination, a linear combination, a non-linear combination, a machine learning output, a parametric combination) of the results from multiple assays, factors, and/or markers. See, e.g., Hamscher, Console and de Kleer (1992) Readings in model-based diagnosis. San Francisco, Calif. (Morgan Kaufmann Publishers Inc.).

In some embodiments, the technology provides one or more machine learning systems (i.e., a machine learning model) for receiving as inputs genetic and/or clinico-genetic data and outputting a classifier of cognitive disease progression and/or predictor of cognitive impairment in a subject. In some embodiments, the machine learning system comprises components for supervised learning and/or unsupervised learning. In some embodiments, the machine learning system comprises a classification component configured to classify subjects using genetic and/or clinico-genetic data obtained from detecting markers in a sample from the subject. In some embodiments, the machine learning system comprises a component configured for decision tree learning, a component configured for association rule learning, a neural network component, a component configured for deep learning, a component configured for inductive logic, a support vector machine component, a cluster analysis component, a Bayesian network component, a component configured for reinforcement learning, a component configured for representation learning, a component configured for similarity and/or metric learning, a component configured for sparse dictionary learning, a component configured to provide a genetic search heuristic algorithm, a component configured to provide rule-based machine learning, and/or a component configured to provide a learning classifier system.

In some embodiments, a machine learning system a machine learning model) comprises a component to validate a machine learning model, e.g., by an accuracy estimation technique. In some embodiments, the accuracy estimation comprises use of the holdout method in which data are split into a training (“reference”) set and test (“external”) set and evaluates the performance of the training model on the test set. In some embodiments, the accuracy estimation comprises use of the N-fold-cross-validation method in which data are randomly split into k subsets and the (k−1) instances of the data are used to train the model while the k^{th} instance is used to test the predictive ability of the training model. In some embodiments, the accuracy estimation comprises use of a bootstrap method in which n instances are sampled with replacement from the dataset.

In one example, a method for characterizing a sample as having been obtained from a human subject having cognitive impairment includes: (a) receiving a sample obtained from the subject; (b) generating input data by detecting, in the sample obtained from the subject, the status of a plurality of markers of cognitive impairment; (c) characterizing a risk for cognitive impairment for the subject using a trained machine learning model configured to receive the generated data and output a cognitive impairment risk assessment for the subject, the trained machine learning model comprising: (i) a plurality of parameters identified using a training data set comprising, for each training sample in the training data set, a status of one or more markers of cognitive impairment and a cognitive impairment status of a subject associated with the training sample; and (ii) a function representing the relation between the status of the one or more markers of cognitive impairment and the cognitive impairment risk assessment; and (d) generating a report characterizing the sample as having been obtained from a human subject having cognitive impairment or having an increased risk of cognitive impairment based on the outputted cognitive impairment risk assessment.

In another example, a method for characterizing plurality of neurodegenerative pathological features of a cognitive impairment in a human subject, comprising: (a) generating first input data by detecting, in a sample obtained from the subject a status of markers in a first panel of markers or markers in linkage disequilibrium with markers in the first panel of markers, wherein the first panel of markers is associated with a first neurodegenerative pathological feature of the cognitive impairment; (b) characterizing a risk for the first neurodegenerative pathological feature for the subject using a first trained machine learning model configured to receive the generated first input data and output a risk assessment for the first neurodegenerative pathological feature for the subject, the first trained machine learning model comprising: (i) a plurality of parameters identified using a first training data set comprising, for each training sample in the first training data set, a status of one or more markers of the first neurodegenerative pathological feature and a first neurodegenerative pathological feature status of a subject associated with the training sample; and (ii) a function representing the relation between the status of the one or more markers of the first neurodegenerative pathological feature and the risk of the first neurodegenerative pathological feature; (c) generating second input data by detecting, in the sample obtained from the subject a status of markers in a second panel of markers or markers in linkage disequilibrium with markers in the second panel of markers, wherein the second panel of markers is associated with a second neurodegenerative pathological feature of the cognitive impairment; (d) characterizing a risk for the second neurodegenerative pathological feature for the subject using a second trained machine learning model configured to receive the generated second input data and output a risk assessment for the second neurodegenerative pathological feature for the subject, the second trained machine learning model comprising: (i) a plurality of parameters identified using a second training data set comprising, for each training sample in the second training data set, a status of one or more markers of the second neurodegenerative pathological feature and a second neurodegenerative pathological feature status of a subject associated with the training sample; and (ii) a function representing the relation between the status of the one or more markers of the second neurodegenerative pathological feature and the risk of the second neurodegenerative pathological feature; and (e) generating a report characterizing the risk of the first neurodegenerative pathological feature and the second neurodegenerative pathological feature based on the output from the first trained machine learning model and the second trained machine learning model.

Some embodiments comprise a storage medium and memory components. Memory, components (e.g., volatile and/or nonvolatile memory) find use in storing instructions (e.g., an embodiment of a process as provided herein) and/or data Some embodiments relate to systems also comprising one or more of a CPU, a graphics card, and a user interface comprising an output device such as display and an input device such as a keyboard). Programmable machines associated with the technology comprise conventional extant technologies and technologies in development or yet to be developed (e.g., a quantum computer, a chemical computer, a DNA computer, an optical computer, a spintronics based computer, etc.). In some embodiments, the technology comprises a wired (e.g., metallic cable, fiber optic) or wireless transmission medium for transmitting data. For example, some embodiments relate to data transmission over a network (e.g., a local area network (LAN), a wide area network (WAN), an ad-hoc network, the internet, etc.). In some embodiments, programmable machines are present on such a network as peers and in some embodiments the programmable machines have a client/server relationship. In some embodiments, data are stored on a computer-readable storage medium such as a hard disk, flash memory, optical media, a floppy disk, etc.

In some embodiments, the technology provided herein is associated with a plurality of programmable devices that operate in concert to perform a method as described herein. For example, in some embodiments, a plurality of computers (e.g., connected by a network) may work in parallel to collect and process data, e.g., in an implementation of cluster computing or grid computing or some other distributed computer architecture that relies on complete computers (with onboard CPUs, storage, power supplies, network interfaces, etc.) connected to a network (private, public, or the internet) by a conventional network interface, such as Ethernet, fiber optic, or by a wireless network technology.

Some embodiments provide a computer that includes a computer-readable medium. The embodiment includes a random access memory (RAM) coupled to a processor. The processor executes computer-executable program instructions stored in memory. Such processors may include a microprocessor, an ASIC, a state machine, or other processor, and can be any of a number of computer processors, such as processors from Intel Corporation of Santa Clara, Calif. and Motorola Corporation of Schaumburg, Ill. Such processors include, or may be in communication with, media, for example computer-readable media, which stores instructions that, when executed by the processor, cause the processor to perform the steps described herein.

Embodiments of computer-readable media include, but are not limited to, an electronic, optical, magnetic, or other storage or transmission device capable of providing a processor with computer-readable instructions. Other examples of suitable media include, but are not limited to, a floppy disk, CD-ROM, DVD, magnetic disk, memory chip, ROM, RAM, an ASIC, a configured processor, all optical media, all magnetic tape or other magnetic media, or any other medium from which a computer processor can read instructions. Also, various other forms of computer-readable media may transmit or carry instructions to a computer, including a router, private or public network, or other transmission device or channel, both wired and wireless. The instructions may comprise code from any suitable computer-programming language, including, for example, C, C++, C# Objective C, Visual Basic, Java, Python, Perl, Swift, Unix, Julia, and JavaScript.

Computers are connected in some embodiments to a network. Computers may also include a number of external or internal devices such as a mouse, a CD-ROM, DVD, a keyboard, a display, or other input or output devices. Examples of computers are personal computers, digital assistants, personal digital assistants, cellular phones, mobile phones, smart phones, pagers, digital tablets, laptop computers, internet appliances, and other processor-based devices. In general, the computers related to aspects of the technology provided herein may be any type of processor-based platform that operates on any operating system, such as Microsoft Windows, Linux, UNIX, macOS, etc., capable of supporting one or more programs comprising the technology provided herein. Some embodiments comprise a personal computer executing other application programs (e.g., applications). The applications can be contained in memory and can include, for example, a word processing application, a spreadsheet application, an email application, an instant messenger application, a presentation application, an Internet browser application, a calendar/organizer application, and any other application capable of being executed by a client device.

All such components, computers, and systems described herein as associated with the technology may be logical or virtual.

The technology finds use in clinical, research, and commercial applications. For instance, in some embodiments, the technology is used to increase the efficiency of recruiting individuals for clinical trials. In some embodiments, the technology is used to provide improved client diagnoses (e.g., identifying patients with cognitive impairment and/or classifying patients having cognitive impairment). In some embodiments, the technology is used to increase the efficiency of researching pharmaceuticals for treating cognitive impairment. In some embodiments, the technology is used to increase the design and production of pharmaceuticals for treating cognitive impairment. In some embodiments, the technology provides an indicator, predictor, and/or classifying that is used to supplement imaging technologies commonly used for identifying cognitive impairment in an individual (e.g., amyloid-PET and/or Tau-PET scans). In some embodiments, the technology provides an indicator, predictor, and/or classifying that is used in lieu of imaging technologies commonly used for identifying cognitive impairment in an individual amyloid-PET and/or Tau-PET scans)).

In some embodiments, the technology described herein provides high quality estimates of pathological processes used to match patients to drugs for trial recruitment. In some embodiments, the technology described herein comprises models to predict disease progression and trajectory relating to estimated pathology load. For instance, using algorithmic feature extraction, models were developed selecting features from the genome (e.g., variants tagging genome-wide association risk loci (e.g., APOE for Alzheimer's disease)) via linkage disequilibrium as well as novel variants of interest tagged in a similar way. In some embodiments, the technology provides a nonlinear combination of predictions using genome-wide data tagging genomic variation (both de novo and known risk factors) and, in some embodiments, clinical and therapeutic data, using machine learning.

### Exemplary Embodiments

The following embodiments are exemplary and are not intended to limit the invention or inventions described herein.

### Embodiment 1

A method for characterizing a plurality of neurodegenerative pathological features of a cognitive impairment in a human subject, comprising:
(a) detecting, in a sample obtained from the subject, a status of first markers in a first panel of markers or markers in linkage disequilibrium with markers in the first panel of markers, wherein the first panel of markers is associated with a first neurodegenerative pathological feature of the cognitive impairment;
(b) detecting, in the same sample obtained from the subject, a status of second markers in a second panel of markers or markers in linkage disequilibrium with markers in the second panel of markers, wherein the second panel of markers is associated with a second neurodegenerative pathological feature of the cognitive impairment; and
(c) characterizing a presence or risk of the first and second neurodegenerative pathological features of the cognitive impairment in the subject based on the status of the first markers and the status of the second markers.

### Embodiment 2

A method of selecting a patient for participation in a clinical trial, comprising:
characterizing a plurality of neurodegenerative pathological features of a cognitive impairment in a human subject according to embodiment 1; and
selecting the patient for participation in the clinical trial based on the characterized presence or risk of the first and second neurodegenerative pathological features of the cognitive impairment in the subject.

### Embodiment 3

The method of embodiment 1 or 2, wherein characterizing the risk of the first and second neurodegenerative pathological features in the subject comprises characterizing a risk that the subject had at the time the sample was obtained from the subject the first neurodegenerative pathological feature, the second neurodegenerative pathological feature, or both.

### Embodiment 4

The method of any one of embodiments 1-3, wherein characterizing the risk of the first and second neurodegenerative pathological features in the subject comprises characterizing a risk that the subject will develop the first neurodegenerative pathological feature, the second neurodegenerative pathological feature, or both.

### Embodiment 5

The method of any one of embodiments 1-4, wherein characterizing the risk of the first and second neurodegenerative pathological features in the subject comprises characterizing a risk that the subject had at the time the sample was obtained from the subject or that the subject will develop the first neurodegenerative pathological feature, the second neurodegenerative pathological feature, or both.

### Embodiment 6

The method of any one of embodiments 1-5, wherein characterizing a risk of the first and second neurodegenerative pathological features in the subject comprises separately characterizing (i) the risk of the first neurodegenerative feature based on the status of the first markers, and (ii) the risk of the second neurodegenerative feature in the subject based on the status of the second markers.

### Embodiment 7

The method of any one of embodiment 1-6, wherein characterizing a risk of the first and second neurodegenerative pathological features in the subject comprises characterizing a composite risk of the first neurodegenerative feature and the second neurodegenerative feature in the subject.

### Embodiment 8

The method any one of embodiments 1-7, wherein characterizing a risk of the first and second neurodegenerative pathological features in the subject comprises characterizing a composite risk of the first neurodegenerative feature or the second neurodegenerative feature in the subject.

### Embodiment 9

The method of any one of embodiments 1-8, wherein detecting a status of first markers or a status of second markers comprises determining the presences or absence of the first markers or the presence or absence of the second markers.

### Embodiment 10

The method of any one of embodiments 1-9, wherein the presence or risk of the first neurodegenerative pathological feature and the presence or risk of the second neurodegenerative pathological feature are characterized using independently selected machine learning systems.

### Embodiment 11

The method of any one of embodiments 1-10, comprising characterizing a presence or risk of three or more neurodegenerative pathological features of the cognitive impairment in the subject using independently selected machine learning systems.

### Embodiment 12

The method of any one of embodiments 1-11, wherein the first neurodegenerative pathological feature and/or the second neurodegenerative pathological feature is amyloid beta, Lewy bodies, tau protein, cerebral amyloid angiopathy (CAA), or a progression of the cognitive impairment.

### Embodiment 13

The method of any one of embodiments 1-12, wherein the first markers and/or the second markers comprise one or more genetic markers.

### Embodiment 14

The method of embodiment 13, wherein the one or more genetic markers comprise one or more functional SNPs and/or one or more tag SNPs.

### Embodiment 15

The method of embodiment 13 or 14, wherein the one or genetic markers comprise one or more of a DNA structural variant, a DNA copy number, a DNA repeat expansion, a DNA short tandem repeat (STR), DNA deletion 20 bases in length or less, a DNA deletion more than 2.1 bases in length, a DNA insertion, an RNA expression level, an RNA SNP, an RNA fusion, an RNA splice variant, or a DNA methylation status.

### Embodiment 16

The method of any one of embodiments 13-15, wherein detecting the status of the genetic marker comprises determining an identity of a nucleotide at a chromosomal location of the genetic marker.

### Embodiment 17

The method of any one of embodiments 1-16, wherein the first markers and/or the second markers comprise clinical markers and/or therapeutic markers.

### Embodiment 18

The method of any one of embodiments 1-17, wherein said markers comprise an APOE allele 2 copy number, APOE allele 4 copy number, biological sex, and/or age.

### Embodiment 19

The method of any one of embodiments 1-18, wherein characterizing the presence or risk of the first and second neurodegenerative pathological features of the cognitive impairment in the subject comprises inputting data describing the status of the first set of markers and/or the second set of markers into one or more machine learning systems.

### Embodiment 20

The method of embodiment 19, wherein the one or more machine learning systems output a predictor of the presence or risk of the first neurodegenerative pathological feature and the presence or risk of the second neurodegenerative pathological feature.

### Embodiment 21

The method of any one of embodiments 1-20, wherein at least the first neurodegenerative pathological feature and the second neurodegenerative pathological feature are used to enroll the subject in a clinical trial.

### Embodiment 22

The method of any one of embodiments 1-21, wherein at least the first neurodegenerative pathological feature and the second neurodegenerative pathological feature are used to determine a course of a treatment for the cognitive impairment.

### Embodiment 23

The method of any one of embodiments 1-22, wherein detecting the status of one or more markers among the first markers or the second markers comprises use of a detection technique selected from the group consisting of microarray analysis, nucleic acid amplification, hybridization analysis, and next generation sequencing.

### Embodiment 24

The method of any one of embodiments 1-23, wherein detecting the status of one or more markers among the first markers or the second markers comprises sequencing nucleic acids from the sample.

### Embodiment 25

A method for characterizing a human subject as having a cognitive impairment, the method comprising:
(a) detecting, in a sample obtained from the subject, the status of markers in a panel of markers or markers in linkage disequilibrium with the markers in the panel of markers; and
(b) characterizing the presence or risk of a cognitive impairment in the subject based on the status of said markers of said panel of markers.

### Embodiment 26

A method for characterizing a human subject as having or at risk for a cognitive impairment, the method comprising:
(a) detecting, in a sample obtained from the subject, the status of markers in a panel of markers or markers in linkage disequilibrium with the markers in the panel of markers; and
(b) characterizing the presence or risk of a cognitive impairment in the subject based on the status of said markers of said panel of markers.

### Embodiment 27

A method of selecting a patient for participation in a clinical trial, comprising:
characterizing the human subject as having a cognitive impairment according to the method of embodiment 25 or 26; and
selecting the patient for participation in the clinical trial based on the characterized presence or risk of the cognitive impairment in the subject.

### Embodiment 28

The method of any one of embodiment 25-27, wherein characterizing the presence or risk of a cognitive impairment in the subject comprising characterizing the risk that the subject had the cognitive impairment at the time the sample was obtained from the subject.

### Embodiment 29

The method of any one of embodiment 25-28, wherein characterizing the presence or risk of a cognitive impairment in the subject comprising characterizing the risk that the subject will develop the cognitive impairment.

### Embodiment 30

The method of any one of embodiment 25-29, wherein characterizing the presence or risk of a cognitive impairment in the subject comprising characterizing the risk that the subject had, at the time the sample was obtained from the subject, or that the subject will develop the cognitive impairment.

### Embodiment 31

The method of any one of embodiments 25-30, wherein detecting the status of markers comprises determining the presence or absence of the markers

### Embodiment 32

The method of any one of embodiments 25-31, wherein characterizing the presence or risk of a cognitive impairment comprises predicting the presence of a neurodegenerative pathological feature.

### Embodiment 33

The method of embodiment 32, wherein the neurodegenerative pathological feature comprises amyloid beta, Lewy bodies, tau protein, cerebral amyloid angiopathy (CAA), or a progression of cognitive impairment.

### Embodiment 34

The method of any one of embodiments 25-33, wherein characterizing the presence or risk of cognitive impairment in the subject comprises inputting data describing the status of said markers of said panel of markers into a machine learning system.

### Embodiment 35

The method of embodiment 34, wherein said machine learning system outputs a predictor of cognitive impairment in the subject.

### Embodiment 36

The method of any one of embodiments 25-35, wherein said markers of said panel of markers comprise one or more genetic markers.

### Embodiment 37

The method of any one of embodiments 25-36, wherein said markers of said panel of markers comprise one or more functional SNPs and/or tag SNPs.

### Embodiment 38

The method of any one of embodiments 25-37, wherein the markers comprise one or more of a DNA structural variant, a DNA copy, number, a DNA repeat expansion, a DNA short tandem repeat (STR), DNA deletion 20 bases in length or less, a DNA deletion more than 21 bases in length, a DNA insertion, an RNA expression level, an RNA SNP, an RNA fusion, an RNA splice variant, or a DNA methylation status.

### Embodiment 39

The method of any one of embodiments 25-38, wherein said markers of said panel of markers comprises one or more clinical markers and/or one or more therapeutic markers.

### Embodiment 40

The method of any one of embodiments 25-39, wherein said markers of said panel of markers comprises APOE allele 2 copy number, APOE allele 4 copy, biological sex, and/or age.

### Embodiment 41

The method of any one of embodiments 25-40, wherein the characterized presence or risk of the cognitive impairment in the subject is used to enroll the human subject in a clinical trial.

### Embodiment 42

The method of any one of embodiments 25-41, wherein the characterized presence or risk of the cognitive impairment in the subject is used to determine the course of a treatment for the human subject.

### Embodiment 43

The method of any one of embodiments 25-42, wherein detecting the status of one or more of the markers in the panel of markers comprises determining the identity of a nucleotide at the chromosomal location of the one or more markers.

### Embodiment 44

The method of any one of embodiments 25-43, wherein detecting the status of one or more of the markers in the panel of markers comprises use of a detection technique selected from the group consisting of microarray analysis, nucleic acid amplification, hybridization analysis, and next generation sequencing.

### Embodiment 45

The method of any one of embodiments 25-44, wherein detecting the status of one or more of the markers in the panel of markers comprises sequencing nucleic acids from the sample.

### Embodiment 46

A method for characterizing a sample as having been obtained from a human subject having cognitive impairment, the method comprising:
(a) receiving a sample obtained from the subject;
(b) detecting, in a sample obtained from the subject, the presence or absence one or more markers of cognitive impairment selected from a panel of markers or markers in linkage disequilibrium with the markers;
(c) using a machine learning system to receive data generated in steps (b) and output a cognitive impairment risk assessment for the human subject from which the sample was obtained; and
(d) characterizing the subject as having a cognitive impairment or having an increased risk of cognitive impairment based on the risk assessment of step (c).

### Embodiment 47

The method of embodiment 46, further comprising identifying said subject as a candidate for a clinical trial.

### Embodiment 48

The method of embodiment 46 or 47, wherein characterizing the subject as having a cognitive impairment or having an increased risk of cognitive impairment comprises predicting the presence of a neurodegenerative pathological feature.

### Embodiment 49

The method of embodiment 48, wherein the pathological feature comprises amyloid beta, Lewy bodies, tau protein, cerebral amyloid angiopathy (CAA), or a progression of cognitive impairment.

### Embodiment 50

The method of any one of embodiments 46-49, wherein characterizing the subject as having a cognitive impairment or having an increased risk of cognitive impairment comprises predicting the presence of more than one pathological feature, where in each pathological feature has a unique set of panel markers.

### Embodiment 51

A method of testing a subject for cognitive impairment, the method comprising:
(a) obtaining a sample from the subject;
(b) providing the sample to a testing facility to be tested for the presence or absence of markers for a panel or markers in linkage disequilibrium with the markers; and
(c) receiving a report from the testing facility indicating presence or risk of cognitive impairment in the subject.

### Embodiment 52

The method of embodiment 51, wherein testing a subject for cognitive impairment comprises predicting the presence of a neurodegenerative pathological feature.

### Embodiment 53

The method of embodiment 52, wherein the neurodegenerative pathological feature comprises amyloid beta, Lewy bodies, tau protein, cerebral amyloid angiopathy (CAA), or a progression of cognitive impairment.

### Embodiment 54

A method for characterizing a human subject as having a cognitive impairment, the method comprising:
(a) detecting; in a sample obtained from the subject, the presence or absence of markers for a panel of markers selected from the markers provided by Table 2 or markers in linkage disequilibrium with the markers in Table 2; and
(b) characterizing the presence or risk of cognitive impairment in the subject based on the presence or absence of said markers of said panel of markers.

### Embodiment 55

The method of embodiment 54, wherein the human subject is suspected of suffering from a cognitive disorder based on the presence of symptoms of a cognitive disorder.

### Embodiment 56

The method of embodiment 54 or 55, wherein the human subject is suspected of suffering from a cognitive disorder based on an assessment of cognitive ability.

### Embodiment 57

The method of embodiment 54; wherein the human subject is suspected of suffering from a cognitive disorder based on a change with time of a score from an assessment of cognitive ability.

### Embodiment 58

The method of any one of embodiments 54-57, wherein characterizing the presence or risk of cognitive impairment in the subject comprises inputting data describing the presence or absence of said markers of said panel of markers into a machine learning system.

### Embodiment 59

The method of embodiment 58, wherein characterizing the presence or risk of cognitive impairment in the subject further comprises inputting data describing clinical and/or therapeutic markers into said machine learning system.

### Embodiment 60

The method of embodiment 59 wherein said clinical and/or therapeutic markers comprise a marker selected from the group consisting of APOE allele 2 copy number, APOE allele 4 copy number, biological sex, and age.

### Embodiment 61

The method of any one of embodiments 58-60, wherein said machine learning system outputs a predictor of cognitive impairment in the subject.

### Embodiment 62

The method of any one of embodiments 58-61, wherein said markers of said panel of markers comprises functional SNPs and/or tag SNPs.

### Embodiment 63

The method of any one of embodiments 58-62, wherein detecting the presence or absence of a marker in the panel of markers comprises determining the identity of a nucleotide at the chromosomal location of said marker.

### Embodiment 64

The method of any one of embodiments 58-63, wherein detecting the presence or absence of a marker in the panel of markers comprises exposing the sample to nucleic acid probes complementary to the genomic sequences corresponding to the markers of the panel.

### Embodiment 65

The method of embodiment 64, wherein the nucleic acid probes are covalently linked to a solid surface.

### Embodiment 66

The method of any one of embodiments 58-65, wherein detecting the presence or absence of a marker in the panel of markers comprises use of a detection technique selected from the group consisting of microarray analysis, nucleic acid amplification, and hybridization analysis.

### Embodiment 67

The method of any one of embodiments 58-66, wherein detecting the presence or absence of a marker in the panel of markers comprises sequencing nucleic acids from the sample.

### Embodiment 68

The method of any one of embodiments 58-67, wherein said panel of markers comprises 5 markers.

### Embodiment 69

The method of any one of embodiments 58-68, wherein said panel of markers comprises 10 markers.

### Embodiment 70

The method of any one of embodiments 58-69, wherein said panel of markers comprises 20 markers.

### Embodiment 71

The method of any one of embodiments 58-70, wherein said panel of markers comprises 50 markers.

### Embodiment 72

A method for classifying progression of cognitive impairment in a human subject, the method comprising:
(a) detecting, in a sample obtained from the subject, the status of markers in a panel of markers or markers in linkage disequilibrium with the markers in the panel of markers; and
(b) classifying progression of cognitive impairment in the human subject based on the status of said markers of said panel of markers.

### Embodiment 73

A method for classifying progression of cognitive impairment in a human subject, the method comprising:
(a) detecting, in a sample obtained from the subject, the presence or absence of markers for a panel of markers selected from the markers provided by Table 1 or markers in linkage disequilibrium with the markers in Table 1; and
(b) classifying progression of cognitive impairment in the human subject based on the presence or absence of said markers of said panel of markers.

### Embodiment 74

The method of embodiment 72 or 73, wherein the human subject is suspected of suffering from a cognitive disorder based on the presence of symptoms of a cognitive disorder.

### Embodiment 75

The method of any one of embodiments 72-74, wherein the human subject is suspected of suffering from a cognitive disorder based on an assessment of cognitive ability.

### Embodiment 76

The method of any one of embodiments 72-75, wherein the human subject is suspected of suffering from a cognitive disorder based on a change with time of a score from an assessment of cognitive ability.

### Embodiment 77

The method of any one of embodiments 72-76, wherein classifying progression of cognitive impairment in said human subject comprises inputting data describing the presence or absence of said markers of said panel of markers into a machine learning system.

### Embodiment 78

The method of embodiment 77, wherein classifying progression of cognitive impairment in said human subject further comprises inputting data describing clinical and/or therapeutic markers into said machine learning system.

### Embodiment 79

The method of embodiment 78, wherein said clinical and/or therapeutic markers comprise a marker selected from the group consisting of APOE allele 4 copy number, APOE allele 2 copy number, biological sex, and age.

### Embodiment 80

The method of any one of embodiments 77-79 wherein said machine learning system outputs a classifier of the progression of cognitive impairment in said human subject.

### Embodiment 81

The method of any one of embodiments 72-80, wherein said markers of said panel of markers comprises functional SNPs and/or tag SNPs.

### Embodiment 82

The method of any one of embodiments 72-81, wherein detecting the presence or absence of a marker, or status of a marker, in the panel of markers comprises determining the identity of a nucleotide at the chromosomal location of said marker.

### Embodiment 83

The method of any one of embodiments 72-82, wherein detecting the presence or absence of a marker, or a status of the marker, in the panel of markers comprises exposing the sample to nucleic acid probes complementary to the genomic sequences corresponding to the markers of the panel.

### Embodiment 84

The method of embodiment 83, wherein the nucleic acid probes are covalently linked to a solid surface.

### Embodiment 85

The method of any one of embodiments 72-84, wherein detecting the presence or absence of a marker in the panel of markers comprises use of a detection technique selected from the group consisting of microarray analysis, nucleic acid amplification, and hybridization analysis.

### Embodiment 86

The method of any one of embodiments 72-85, wherein detecting the presence or absence of a marker in the panel of markers comprises sequencing nucleic acids from the sample.

### Embodiment 87

The method of any one of embodiments 72-86, wherein said panel of markers comprises 5 markers.

### Embodiment 88

The method of any one of embodiments 72-87, wherein said panel of markers comprises 10 markers.

### Embodiment 89

The method of any one of embodiments 72-88, wherein said panel of markers comprises 20 markers.

### Embodiment 90

The method of any one of embodiments 72-89, wherein said panel of markers comprises 50 markers.

### Embodiment 91

A method for characterizing a sample as having been obtained from a human subject having cognitive impairment, the method comprising:
(a) receiving a sample obtained from the subject;
(b) generating input data by detecting, in a sample obtained from the subject, the status of a plurality of markers of cognitive impairment;
(c) using a trained machine learning model configured to receive the generated data and output a cognitive impairment risk assessment for the human subject from which the sample was obtained, the trained machine learning model comprising:
   (i) a plurality of parameters identified using a training data set comprising, for each training sample in the training data set, a status of one or more markers of cognitive impairment and a cognitive impairment status of a subject associated with the training sample; and
   (ii) a function representing the relation between the status of the one or more markers of cognitive impairment and the cognitive impairment risk assessment; and
(d) generating a report characterizing the sample as having been obtained from a human subject having cognitive impairment or having an increased risk of cognitive impairment based on the outputted cognitive impairment risk assessment.

### Embodiment 92

A method for characterizing a sample as having been obtained from a human subject having cognitive impairment, the method comprising:
(a) receiving a sample obtained from the subject;
(b) detecting, in a sample obtained from the subject, the presence or absence of a first marker of cognitive impairment selected from the markers provided by Table 2 or in linkage disequilibrium with a marker provided by Table 2;
(c) detecting, in said sample; the presence or absence of a second marker of cognitive impairment selected from the markers provided by Table 2 or in linkage disequilibrium with a marker provided by Table 2;
(d) using a machine learning system to receive data generated in steps (h) and (c) and output a cognitive impairment risk assessment for the human subject from which the sample was obtained; and
(e) generating a report characterizing the sample as having been obtained from a human subject having cognitive impairment or having an increased risk of cognitive impairment based on the risk assessment of step (d).

### Embodiment 93

The method of embodiment 92 or 93 further comprising identifying said subject as a candidate for a clinical trial.

### Embodiment 94

A method for classifying progression of cognitive impairment in a human subject, the method comprising:
(a) receiving a sample obtained from the subject;
(b) detecting, in a sample obtained from the subject, the presence or absence of a first marker of cognitive impairment selected from the markers provided by Table 1 or in linkage disequilibrium with a marker provided by Table 1;
(c) detecting, in said sample, the presence or absence of a second marker of cognitive impairment selected from the markers provided by Table 1 or in linkage disequilibrium with a marker provided by Table 1;
(d) using a machine teaming system to receive data generated in steps (b) and (c) and output a cognitive impairment progression classifier for the human subject from which the sample was obtained; and
(e) generating a report classifying the progression of cognitive impairment in the human subject based on the risk assessment of step (d).

### Embodiment 95

The method of embodiment 94, further comprising identifying said subject as a candidate for a clinical trial.

### Embodiment 96

A method of testing a subject for cognitive impairment, the method comprising:
(a) obtaining a sample from the subject;
(b) providing the sample to testing facility to be tested for the presence or absence of markers for a panel of markers selected from the markers provided by Table 2 or markers in linkage disequilibrium with the markers in Table 2; and
(c) receiving a report from the testing facility indicating presence or risk of cognitive impairment in the subject.

### Embodiment 97

A method of classifying progression of cognitive impairment in a human subject, the method comprising:
(a) obtaining a sample from the subject;
(b) providing the sample to testing facility to be tested for the presence or absence of markers for a panel of markers selected from the markers provided by Table 1 or markers in linkage disequilibrium with the markers in Table and
(c) receiving a report from the testing facility classifying progression of cognitive impairment in the human subject.

### Embodiment 98

The method of any one of embodiments 1-97, wherein the cognitive impairment is associated with Alzheimer's disease or dementia.

### Embodiment 99

Use of one or more marker panels or markers in linkage disequilibrium with the markers to test a subject for cognitive impairment.

### Embodiment 100

Use of a marker panel comprising markers provided by Table 2 or markers in linkage disequilibrium with the markers in Table 2 to test a subject for cognitive impairment.

### Embodiment 101

Use of a marker panel comprising markers provided by Table 1 or markers in linkage disequilibrium with the markers in Table 1 to classify progression of cognitive impairment in a human subject.

### Embodiment 102

A kit, reagent mixture, or surface comprising reagents for detecting a panel comprising multiple markers listed in Table 1 or Table 2 or markers in linkage disequilibrium with markers listed in Table 1 or Table 2.

### Embodiment 103

A kit, reagent mixture, or surface of embodiment 102, comprising reagents for detection of 1000 or fewer markers.

### Embodiment 104

A kit, reagent mixture, or surface of embodiment 102 or 103, comprising reagents for detection of 5 or more markers listed in Table 1 or Table 2 or markers in linkage disequilibrium with markers listed in Table 1 or Table 2.

### Embodiment 105

A kit, reagent mixture, or surface of any one of embodiments 102-104, comprising reagents for detection of 10 or more markers listed in Table 1 or Table 2 or markers in linkage disequilibrium with markers listed in Table 1 or Table 2.

### Embodiment 106

A kit, reagent mixture, or surface of any one of embodiments 102 105, comprising reagents for detection of 20 or more markers listed in Table 1 or Table 2 or markers in linkage disequilibrium with markers listed in Table 1 or Table 2.

### Embodiment 107

A kit, reagent mixture, or surface of any one of embodiments 102-106, comprising reagents for detection of 50 or more markers listed in Table 1 or Table 2 or markers in linkage disequilibrium with markers listed in Table 1 or Table 2.

### Embodiment 108

A method for characterizing plurality of neurodegenerative pathological features of a cognitive impairment in a human subject, comprising:
(a) generating first input data by detecting, in a sample obtained from the subject a status of markers in a first panel of markers or markers in linkage disequilibrium with markers in the first panel of markers, wherein the first panel of markers is associated with a first neurodegenerative pathological feature of the cognitive impairment;
(b) characterizing a risk for the first neurodegenerative pathological feature for the subject using a first trained machine learning model configured to receive the generated first input data and output a risk assessment for the first neurodegenerative pathological feature for the subject, the first trained machine learning model comprising:
   (i) a plurality of parameters identified using a first training data set comprising, for each training sample in the first training data set, a status of one or more markers of the first neurodegenerative pathological feature and a first neurodegenerative pathological feature status of a subject associated with the training sample; and
   (ii) a function representing the relation between the status of the one or more markers of the first neurodegenerative pathological feature and the risk of the first neurodegenerative pathological feature;
(c) generating second input data by detecting, in the sample obtained from the subject a status of markers in a second panel of markers or markers in linkage disequilibrium with markers in the second panel of markers, wherein the second panel of markers is associated with a second neurodegenerative pathological feature of the cognitive impairment;
(d) characterizing a risk for the second neurodegenerative pathological feature for the subject using a second trained machine learning model configured to receive the generated second input data and output a risk assessment for the second neurodegenerative pathological feature for the subject, the second trained machine learning model comprising:
   (i) a plurality of parameters identified using a second training data set comprising, for each training sample in the second training data set, a status of one or more markers of the second neurodegenerative pathological feature and a second neurodegenerative pathological feature status of a subject associated with the training sample; and
   (ii) a function representing the relation between the status of the one or more markers of the second neurodegenerative pathological feature and the risk of the second neurodegenerative pathological feature; and
(e) generating a report characterizing the risk of the first neurodegenerative pathological feature and the second neurodegenerative pathological feature based on the output from the first trained machine learning model and the second trained machine learning model.

### Embodiment 109

A method of selecting a patient for participation in a clinical trial, comprising:
characterizing a plurality of neurodegenerative pathological features of a cognitive impairment in a human subject according to embodiment 108; and
selecting the patient for participation in the clinical trial based on the characterized risk of the first and second neurodegenerative pathological features of the cognitive impairment in the subject.

### Embodiment 110

The method of embodiment 108 or 109, wherein characterizing the risk of the first neurodegenerative pathological feature and the second neurodegenerative pathological feature comprises characterizing a risk that the subject had at the time the sample was obtained from the subject the first neurodegenerative pathological feature, the second neurodegenerative pathological feature, or both.

### Embodiment 111

The method of any one of embodiments 108-110, wherein characterizing the risk of the first neurodegenerative pathological feature and the second neurodegenerative pathological feature comprises characterizing a risk that the subject will develop the first neurodegenerative pathological feature, the second neurodegenerative pathological feature, or both.

### Embodiment 112

The method of any one of embodiments 108-111, wherein characterizing the risk of the first neurodegenerative pathological feature and the second neurodegenerative pathological feature comprises characterizing a risk that the subject had at the time the sample was obtained from the subject or that the subject will develop the first neurodegenerative pathological feature, the second neurodegenerative pathological feature, or both.

### Embodiment 113

The method of any one of embodiments 108-112, wherein characterizing the risk of the first neurodegenerative pathological feature and the second neurodegenerative pathological feature comprises characterizing a composite risk of the first neurodegenerative feature and the second neurodegenerative feature in the subject.

### Embodiment 114

The method any one of embodiments 108-113, wherein characterizing the risk of the first neurodegenerative pathological feature and the second neurodegenerative pathological feature comprises characterizing a composite risk of the first neurodegenerative feature or the second neurodegenerative feature in the subject.

### Embodiment 115

The method of any one of embodiments 108-114, wherein detecting the status of markers in the first panel or the status of markers in the second panel comprises determining the presences or absence of the markers in the first panel or the presence or absence of markers in the second panel.

### Embodiment 116

The method of any one of embodiments 108-115, wherein first machine learning model and the second machine learning model are independently selected.

### Embodiment 117

The method of any one of embodiments 108-116, comprising characterizing a risk of three or more neurodegenerative pathological features of the cognitive impairment in the subject using independently selected machine learning systems.

### Embodiment 118

The method of any one of embodiments 108-117, wherein the first neurodegenerative pathological feature and/or the second neurodegenerative pathological feature is amyloid beta, Lewy bodies, tau protein, cerebral amyloid angiopathy (CAA), or a progression of the cognitive impairment.

### Embodiment 119

The method of any one of embodiments 108-118, wherein the markers of the first panel and/or the markers of the second panel comprise one or more genetic markers.

### Embodiment 120

The method of embodiment 119, wherein the one or more genetic markers comprise one or more functional SNPs and/or one or more tag SNPs.

### Embodiment 121

The method of embodiment 119 or 120, wherein the one or genetic markers comprise one or more of a DNA structural variant, a DNA copy number, a DNA repeat expansion, a DNA short tandem repeat (STR), DNA deletion 20 bases in length or less, a DNA deletion more than 21 bases in length, a DNA insertion, an RNA expression level, an RNA SNP, an RNA fusion, an RNA splice variant, or a DNA methylation status.

### Embodiment 122

The method of any one of embodiments 119-121, wherein detecting the status of the genetic marker comprises determining an identity of a nucleotide at a chromosomal location of the genetic marker.

### Embodiment 123

The method of any one of embodiments 108-122, wherein the first markers and/or the second markers comprise clinical markers and/or therapeutic markers.

### Embodiment 124

The method of any one of embodiments 108-123, wherein said markers comprise an APOE allele 2 copy number, APOE allele 4 copy number, biological sex, and/or age.

### Embodiment 125

The method of any one of embodiments 108-124, further comprising enrolling the subject in a clinical trial based on the risk of the first neurodegenerative pathological feature and the second neurodegenerative pathological feature.

### Embodiment 126

The method of any one of embodiments 108-125, wherein at least the first neurodegenerative pathological feature and the second neurodegenerative pathological feature are used to determine a course of a treatment for the cognitive impairment.

### Embodiment 127

The method of any one of embodiments 108-126, wherein detecting the status of one or more markers among the markers of the first panel or the markers of the second panel comprises use of a detection technique selected from the group consisting of microarray analysis, nucleic acid amplification, hybridization analysis, and next generation sequencing.

### Embodiment 128

The method of any one of embodiments 108-127, wherein detecting the status of one or more markers among the first markers or the second markers comprises sequencing nucleic acids from the sample

### Example

During the development of embodiments of the technology provided herein, experiments were conducted to develop a machine learning system to identify genetic markers and, optionally, clinical and/or therapeutic data indicative of neuropathologies in brain samples. Pathology data were generated using genotyping arrays to evaluate approximately 1,000 to 1,500 reference brain samples that were pathologically characterized and known to comprise neurodegenerative pathological features (e.g., tau protein, amyloid beta, cerebral amyloid angiopathy (CAA), and/or Lewy bodies). Further, clinical data describing the reference brain samples were also collected.

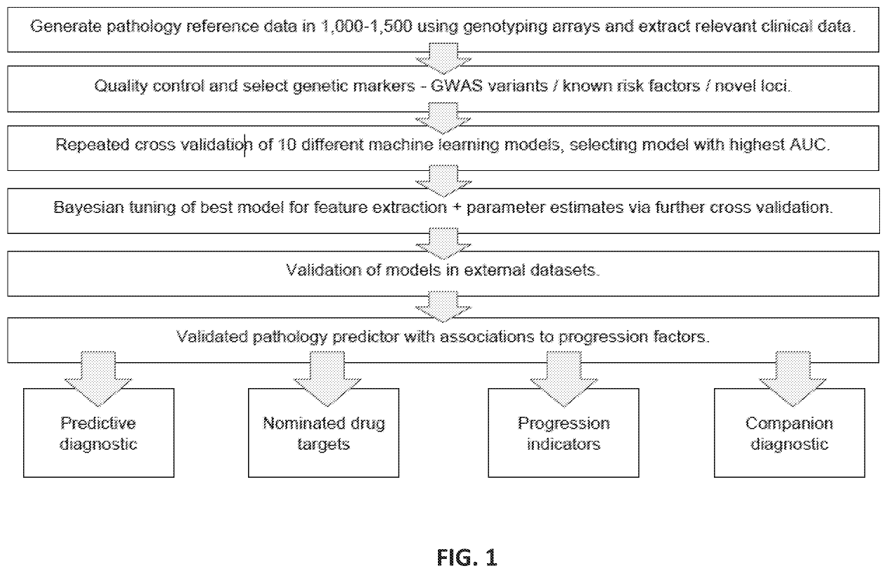

Genetic markers (and, optionally, clinical markers) were selected from the input data and used to produce a pathology predictor using a series of components of the machine learning system including, e.g., an input data quality control component, an input variant selection component, a model selection component, a statistical tuning component, a parameter extraction component, a validation component, and a predictor output component (see, FIG. 1).

Input data (e.g., genetic marker data, clinical data, and/or therapeutic data) were selected by the input data quality control component and/or input variant selection component from GWAS variants, known risk factors, and novel loci identified from the genotyping array data produced from the reference samples. The model selection component cross-validated multiple machine learning models using the input data to select the best model indicative of the known pathologies in the reference samples. For example, some experiments performed repeated cross-validation of 10 different machine learning models and selected the model with the highest area under the receiver operating characteristic curve plotting the true positive rate versus the false positive rate. The statistical tuning component tuned the model selected by the model selection component and the parameter extraction component estimated parameters for the selected model. For example, some experiments used a statistical tuning component that applied Bayesian tuning to the selected model and some experiments used a validation component that applied cross-validation to estimate the parameters for the model. The validation component validated the selected model (e.g., the statistically tuned model comprising the estimated parameters) using datasets that were external to the reference dataset. The predictor output component produced a validated pathology predictor indicative of the presence of pathological factors. Further, in some embodiments, the predictor output component produced a classifier that classified the pathological factors and/or samples based on progression of disease. The pathology predictor and/or classifier finds use as a predictive diagnostic, as a companion diagnostic, to nominate drug targets, and/or to indicate disease progression (see, e.g., FIG. 1).

During the development of embodiments of the technology, experiments were conducted according to the following procedures and methods:

### Input Data Quality Control and Variant Selection

Standard genome-wide association study (GWAS) quality control was implemented for this analysis. Inclusion criteria for samples included: concordance between genotype estimated and reported biological sex, sample call rate >95%, no cryptically related or otherwise related samples at the first cousin level or closer (>12.5% proportional sharing of genotypes), no heterozygosity outliers (|F statistic|<10%), and confirmed European ancestry using the 1000 Genomes Project non-Finnish Europeans as a reference (<|6| SD from mean for principal components 1 and 2) (see, e.g., “A global reference for human genetic variation (2015) Nature 526: 68, incorporated herein by reference). Inclusion criteria for variants included: genotype call rate >95%, Hardy-Weinberg equilibrium P-value >1×10⁻⁵, non-random missingness by case-control status or haplotype P>1×10⁻⁵ and minor allele frequency >5%. All data management, quality control, and analyses were carried out utilizing Rv3.5 (see, e.g., R Core Team (2013) “R: A language and environment for statistical computing” R Foundation for Statistical Computing, Vienna, Austria, incorporated herein by reference) and/or PLINKv1.91,2 (see, e.g., Chang et al. (2015) “Second-generation PLINK: rising to the challenge of larger and richer datasets” Gigascience 4: 7, incorporated herein by reference).

Externally validated and concordant APOE genotypes were merged in to the dataset after quality control. The APOE gene encodes the apolipoprotein E protein, which is a protein that combines with lipids in the body to form lipoproteins. APOE is found on chromosome 19 (19q13.32) at bases 45,409,011 to 45,412,650 (GRCh37). APOE has 3 alleles referred to by the terms “e2” (or “2”), “e3” (or “3”), and “e4” (or “4”) that produce the E2, E3, and E4 isoforms of the ApoE protein. The e3 allele is most common in the general population. The E2 (OMIM entry 107741.0001), E3 (OMIM entry 107741.0015), and E4 (OMIM entry 107741.0016) isoforms differ in amino acid sequence at 2 sites, residue 112 (called site A) and residue 158 (called site B). At sites A/B, ApoE2, Apo-E3, and Apo-E4 contain cysteine/cysteine, cysteine/arginine, and arginine/arginine, respectively. The SNP for the e2 allele is found on chromosome 19 at nucleotide 45412008 (Assembly GRCh37). The SNP for the e3 allele is found on chromosome 19 at nucleotide 45411902 (Assembly GRCh37). The SNP for the e4 allele is found on chromosome 19 at nucleotide 45411941 (Assembly GRCh37).

### Machine Learning Pipeline

The machine learning pipeline began with SNP selection using the R package PRSICIEv23 (see, e.g., Euesden et al., PRSice: Polygenic Risk Score software, Bioinformatics vol. 31, p. 1466 (2015), incorporated herein by reference). Permutation testing and p-value aware LD pruning were used to identify optimal P thresholds for variant inclusion below genome-wide significance levels (GWAS P<5×10⁻⁸, incorporating most recent AD GWAS summary statistics available). LD parameters for variant exclusion related to sliding window sizes of 250 kb, removing variants within these windows at r²>0.1. For each dataset, 10,000 permutations were used to generate empirical P estimates for each GWAS derived P threshold ranging from 5×10⁻⁸ to 0.5, by increments of 5×R² values were estimated between the additive genetic risk scores constructed at these steps and the outcomes amyloid positive status or annual rate of MMSE decline) and adjusted for an estimated prevalence of AD and eigenvectors 1-5 from principal components, age, and sex as emanates (Nagelkerke's pseudo r² was used for amyloid positive status).

Permutation tests identified sets of variants that were most informative in additive linear combinations as predictors of MMSE decline or amyloid status. These analyses provided variant lists for further more powerful analyses using the R package CARET for testing a variety of machine learning models per trait. For the continuous measure of MMSE decline, the following models were tested: glm, bayesglm, xgbTree, xgbDART, xgbLinear, rf, ridge, evtree, glmnet, svmRadial, earth, and lasso. For the dichotomous indicator of amyloid+/− status, the following models were tested: glm, bayesglm, xgbTree, xgbDART, rf, nb, nnet, dim, C5.0, glmnet, svmRadial, and Ida. These initial models underwent a 30× grid search for tuning parameters at a 10×10 repeated cross-validation phase. For each series of models, the best performing model was identified based on either mean r² or mean AUC maximization where appropriate at the training phase during the 10×10 repeated cross validation. After the best performing model at cross-validation was defined, the selected model underwent an additional 100 iterations of Bayesian optimization to tune hyperparameters further (see, e.g., Yachen Yan “A Pure R implementation of Bayesian Global Optimization with Gaussian Processes” available at http://github.com/yanyachen/rBayesianOptimization rBayesianOptimization, incorporated herein by reference). Each optimized model was then fit to the withheld, external test datasets for validation.

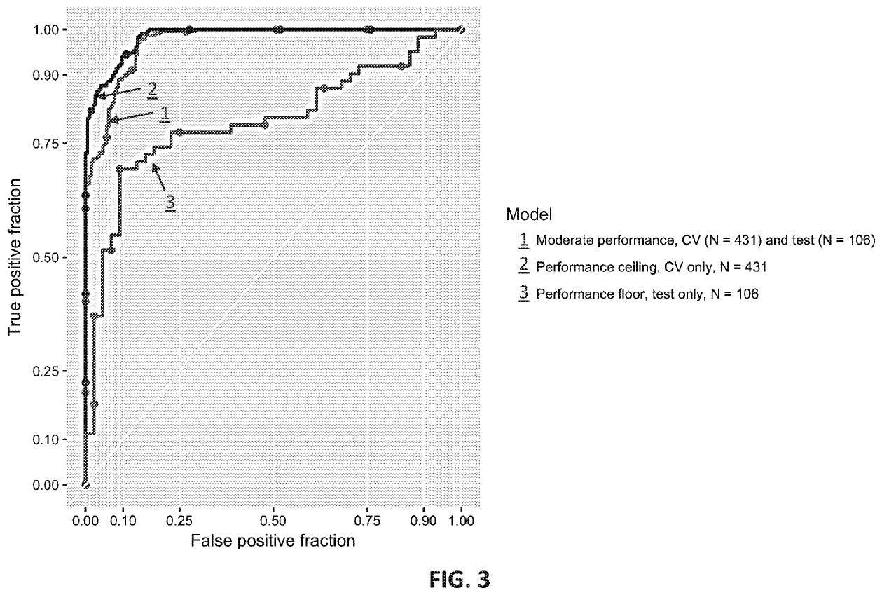

FIG. 3 shows the ROC describing the performance of an embodiment of a machine learning predictor/classifier according to the technology described herein. As shown in FIG. 3, the performance floor (lower trace), performance ceiling (higher trace), and moderate performance curves (intermediate trace), are all significantly shifted toward the upper left corner, indicating high sensitivity and high specificity (e.g., minimizing false negatives and minimizing false positives). Furthermore, during the development of embodiments of the technology, data collected indicated that the predictive values produced were in the range of 70-99% area under the curve for pathological features (e.g., amyloid, tau, and Lewy burdens in the brain). In some embodiments of the technology, models for predicting increased amyloid burden were associated with more rapid decrease in mini mental state examination tests (MMSE, p<1×10⁻⁵) among other markers of progression.

### Markers for Classifying Disease Progression

During the development of embodiments of the technology, experiments were conducted in which the machine learning system identified genetic markers and clinical and/or therapeutic markers for classifying disease progression, e.g., indicative of decreases in cognitive impairment (e.g., as assessed by MMSE score) with time.

The genetic markers and clinical and/or therapeutic markers that were identified are provided in Table 1. In Table 1, the genetic markers are designated at genomic loci (single nucleotide positions) within the Genome Reference Consortium Human Build 37 (GRCh37, Feb. 27, 2009, available at the NCBI at GenBank assembly accession number GCA_000001405.1 and RefSeq assembly accession: GCF_000001405.13) and are indicated using: 1) the human chromosome number designated by the abbreviation “chr” followed by the chromosome number; and 2) the nucleotide position of a SNP identified by the machine learning system. Clinical and/or therapeutic markers are Age, Biological Sex (e.g., female), APOE allele 4 copy number, APOE allele 2 copy number.

TABLE 1
Example set of markers for classifying
cognitive disease progression

| | | |
|---|---|---|
| chr19:45387596 | chr19:45201694 | chr5:153676440 |
| chr19:45416478 | chr7:143107876 | chr7:99696797 |
| chr19:45329214 | chr6:32388275 | chr15:50992311 |
| chr19:45412079 | chr2:234075691 | chr11:65653242 |
| chr19:45384931 | chr10:11719074 | chr11:85716032 |
| chr19:45463386 | chr19:45052601 | chr19:45286639 |
| chr19:45655333 | chr17:5233817 | chr2:127829282 |
| chr19:45237812 | chr11:121451813 | chr17:56404349 |
| chr19:45379060 | chr6:41129207 | chr17:1444702 |
| chr2:127892810 | chr20:55018260 | chr19:1057137 |
| chr19:45165912 | chr18:56189459 | chr1:161116022 |
| chr19:45483438 | chr19:45830947 | chr10:61665886 |
| chr19:45708758 | chr16:31122571 | chr16:30030195 |
| chr8:27464519 | chr4:11025131 | chr15:79231478 |
| chr1:207784968 | chr6:32681277 | chr7:37836588 |
| chr19:45383830 | chr8:27373865 | chr1:161159147 |
| chr11:85867875 | chr17:61560763 | chr19:45086946 |
| chr19:45146103 | chr7:99702947 | chr7:1590280 |
| chr19:45299199 | chr14:92926952 | chr19:45496303 |
| chr19:45370941 | chr2:127812256 | chr7:143127771 |
| chr7:99971313 | chr17:47428573 | chr17:4763551 |
| chr11:59945745 | chr21:27534261 | chr19:45245015 |
| chr19:45338895 | chr15:63553994 | chr11:121448972 |
| chr8:27226790 | chr7:100013402 | chr6:41154650 |
| chr19:45728059 | chr8:103584064 | chr2:127837041 |
| chr19:45461996 | chr19:1046520 | chr9:95845152 |
| chr6:47432637 | chr19:45962799 | chr16:81773209 |
| chr19:1039444 | chr3:155314034 | chr16:81824242 |
| chr6:32376176 | chr16:70666410 | chr16:81912580 |
| chr6:32681483 | chr19:45371168 | chr2:184405092 |
| chr14:92938415 | chr11:47449072 | chr16:30809063 |
| chr15:59034174 | chr2:233977318 | chr14:53400629 |
| chr19:51727962 | chr4:112987361 | chr5:176952919 |
| chr14:32949330 | chr19:51681965 | chr8:144995964 |
| chr14:92993336 | chr1:237931094 | chr19:46146762 |
| chr8:101671221 | chr10:18789498 | chr1:207823240 |
| chr1:207441975 | chr19:45409579 | chr13:80843549 |
| chr2:135597628 | chr19:45589595 | chr2:106383390 |
| chr7:103987785 | chr7:111580166 | chr6:46006950 |
| chr19:45163671 | chr8:27560651 | chr19:5142473 |
| chr17:20657846 | chr8:1681733 | chr7:130616236 |
| chr5:105772632 | chr14:106009572 | chr6:32367515 |
| chr19:46322585 | chr17:42430244 | chr14:29087550 |
| chr2:127846505 | chr6:47889051 | chr19:44286513 |
| chr19:1013634 | chr6:31134888 | chr10:56015656 |
| chr5:86187316 | chr7:37885121 | chr6:80431894 |
| chr2:198186086 | chr2:37540441 | chr19:49218060 |
| chr19:41098691 | chr7:12275818 | chr20:391025 |
| chr19:54816509 | chr17:53166323 | chr7:47386422 |
| chr17:5019668 | chr19:45097027 | chr3:6695911 |
| chr16:55752724 | chr3:182802874 | chr10:82043226 |
| chr6:47704736 | chr11:34578340 | chr15:38350008 |
| chr6:32627714 | chr6:114456597 | chr17:72811343 |
| chr16:8875529 | chr15:58680643 | chr19:5132475 |
| chr1:161012760 | chr15:77284160 | chr13:93477311 |
| chr7:129333905 | chr8:65476548 | chr19:50451508 |
| chr11:85878905 | chr19:45600991 | chr6:114456335 |
| chr1:81316894 | chr7:47195053 | chr9:1218816 |
| chr5:6845035 | chr6:22307725 | chr1:66392405 |
| chr10:124165615 | chr8:27759126 | chr4:185407530 |
| chr14:92863359 | chr19:45039852 | chr17:17698254 |
| chr6:32191339 | chr6:32798548 | chr8:139353715 |
| chr4:14026281 | chr4:159729794 | chr3:178147392 |
| chr20:36207473 | chr8:78510649 | chr7:8101099 |
| chr11:3679811 | chr6:41219627 | chr12:339320 |
| chr12:94934823 | chr1:21180181 | chr12:62423566 |
| chr13:104559991 | chr2:18681809 | chr2:161242295 |
| chr5:58668132 | chr9:8271941 | chr4:73413223 |
| chr8:20986072 | chr13:98400230 | chr19:1090803 |
| chr5:160359601 | chr2:65100346 | chr13:31605117 |
| chr1:30662538 | chr12:108038203 | chr5:88223420 |
| chr15:93600976 | chr10:94147345 | chr15:101767290 |
| chr2:44253448 | chr10:88413432 | chr9:91768648 |
| chr12:32704037 | chr20:40324368 | chr9:9272841 |
| chr5:154749572 | chr1:63912182 | chr13:88622740 |
| chr17:71753573 | chr19:45385389 | chr11:62688269 |
| chr5:35353087 | chr14:50844119 | chr21:41603434 |
| chr6:19434157 | chr17:61557773 | chr19:55176262 |
| chr10:25247006 | chr16:54100006 | chr1:66830107 |
| chr8:95973465 | chr2:214917099 | chr4:112371633 |
| chr10:43341976 | chr19:5037083 | chr12:118343416 |
| chr9:10691050 | chr12:94516195 | chr5:55780101 |
| chr15:50508305 | chr11:85623607 | chr4:99877445 |
| chr3:57303684 | chr9:86214149 | chr5:160264007 |
| chr4:160249722 | chr7:146254508 | chr14:30137407 |
| chr2:71525698 | chr2:72319253 | chr16:90024206 |
| chr18:65062843 | chr3:4951084 | chr22:44745583 |
| chr14:23403171 | chr16:22883851 | chr19:44461049 |
| chr21:15894278 | chr14:25526309 | chr10:86467133 |
| chr3:167132319 | chr6:135233909 | chr3:21467663 |
| chr3:154756700 | chr1:21525228 | chr18:69900152 |
| chr7:28998663 | chr12:100192515 | chr16:16899495 |
| chr8:133637659 | chr4:82752721 | chr5:168549524 |
| chr11:122868205 | chr6:28201138 | chr21:46232921 |
| chr17:49386033 | chr4:61788197 | chr5:150505892 |
| chr5:141883061 | chr6:11513834 | chr6:47596016 |
| chr6:52190041 | chr8:126584451 | chr7:50305863 |
| chr5:139805611 | chr17:42116056 | chr12:75178531 |
| chr7:143192104 | chr14:92876837 | chr6:109503976 |
| chr1:241833287 | chr4:45626526 | chr3:143571147 |
| chr10:98026075 | chr3:135291759 | chr6:133517005 |
| chr15:99066347 | Age | APOE allele 4 copy |
| | | number |
| chr17:260747 | Biological Sex | APOE allele 2 copy |
| | | number |

### Markers Indicating Neurodegenerative Pathology

During the development of embodiments of the technology, experiments were conducted in which the machine learning system identified genetic markers and clinical and/or therapeutic markers for indicating the presence of a neurodegenerative pathology in a subject, e.g., indicative of Alzheimer's Disease (e.g., indicative of tau protein, amyloid beta, cerebral amyloid angiopathy (CAA), and/or Lewy bodies).

The genetic markers and clinical and/or therapeutic markers that were identified are provided in Table 2. In Table 2, the genetic markers are designated at genomic loci (single nucleotide positions) within the Genome Reference Consortium Human Build 37 (GRCh37, Feb. 27, 2009, available at the NCBI at GenBank assembly accession number GCA_000001405.1 and RefSeg assembly accession: GCF_000001405.13) and are indicated using: I) the human chromosome number designated by the abbreviation “chr” followed by the chromosome number; 2) nucleotide position of a SNP identified by the machine learning system to be indicative of the presence of a neurodegenerative pathology in a subject; and 3) the nucleotide base at the specified nucleotide position that is indicative of the presence of a neurodegenerative pathology in a subject. Clinical and/or therapeutic markers are Age, Biological Sex (e.g., female), APOF allele 4 copy number, and APOF allele 2 copy number.

TABLE 2
Example Markers indicating neurodegenerative pathology

| | | |
|---|---|---|
| APOE allele 4 copy number | chr7:37836588_G | chr15:63553994_T |
| chr1:81316894_C | chr7:99702947_A | chr16:30030195_C |
| chr1:207441975_G | chr7:111580166_C | chr16:70666410_A |
| chr2:127812256_A | chr8:1681733_A | chr17:1444702_T |
| chr2:127892810_T | chr8:27560651_T | chr17:20657846_A |
| chr2:233977318_A | chr8:103584064_G | chr17:56404349_G |
| chr4:11025131_C | chr10:61665886_A | chr19:1039444_C |
| chr5:86187316_T | chr11:47449072_A | chr19:45039852_A |
| chr6:22307725_{——}T | chr11:85867875_A | chr19:45146103_C |
| chr6:32388275_C | chr12:94934823_G | chr19:45237812_C |
| chr6:41129207_T | chr14:92863359_C | chr19:45329214_G |
| chr6:47889051_C | chr14:106009572_T | chr19:45379060_C |
| chr19:45409579_T | chr17:42430244_T | chr14:32949330_G |
| chr19:45463386_A | chr17:61560763_T | chr14:92938415_T |
| chr19:45600991_C | chr19:1046520_G | chr15:58680643_T |
| chr19:45830947_A | chr19:45052601_T | chr15:79231478_C |
| chr19:51727962_A | chr19:45163671_C | chr16:31122571_T |
| chr21:27534261_C | chr19:45245015_A | chr16:81824242_G |
| APOE allele 2 copy number | chr19:45338895_A | chr17:5019668_C |
| chr1:161012760_C | chr19:45383830_T | chr17:47428573_C |
| chr1:207784968_A | chr19:45412079_T | chr18:56189459_C |
| chr2:127829282_T | chr19:45483438_C | chr19:1057137_A |
| chr2:135597628_G | chr19:45655333_C | chr19:45086946_A |
| chr2:234075691_A | chr19:45962799_G | chr19:45165912_A |
| chr4:14026281_G | chr19:54816509_T | chr19:45286639_G |
| chr5:105772632_T | Age | chr19:45370941_T |
| chr6:31134888_T | chr1:161116022_T | chr19:45384931_A |
| chr6:32627714_T | chr1:237931094_T | chr19:45416478_A |
| chr6:41154650_T | chr2:127837041_T | chr19:45496303_T |
| chr6:114456597_A | chr2:184405092_T | chr19:45708758_G |
| chr7:37885121_T | chr3:155314034_A | chr19:46322585_T |
| chr7:99971313_T | chr4:112987361_G | chr20:36207473_G |
| chr7:129333905_A | chr5:153676440_T | Biological Sex |
| chr8:27226790_C | chr6:32191339_T | chr1:161159147_T |
| chr8:27759126_T | chr6:32681277_G | chr2:37540441_C |
| chr9:95845152_T | chr6:47432637_C | chr2:127846505_C |
| chr10:124165615_A | chr7:1590280_A | chr2:198186086_A |
| chr11:59945745_C | chr7:47195053_T | chr3:182802874_T |
| chr11:85878905_T | chr7:100013402_T | chr5:6845035_C |
| chr13:104559991_A | chr7:143107876_T | chr5:176952919_C |
| chr14:92926952_T | chr8:27373865_A | chr6:32376176_T |
| chr15:50992311_A | chr8:65476548_T | chr6:32681483_C |
| chr15:77284160_G | chr10:11719074_G | chr6:47704736_T |
| chr16:30809063_A | chr11:3679811_T | chr7:12275818_G |
| chr16:81773209_A | chr11:65653242_T | chr7:99696797_C |
| chr17:4763551_C | chr11:121448972_C | chr7:103987785_T |
| chr7:143127771_G | chr15:59034174_T | chr19:45201694_T |
| chr8:27464519_T | chr16:8875529_T | chr19:45299199_T |
| chr8:101671221_C | chr16:55752724_A | chr19:45371168_A |
| chr10:18789498_G | chr16:81912580_T | chr19:45387596_A |
| chr11:34578340_G | chr17:5233817_T | chr19:45461996_G |
| chr11:85716032_T | chr17:53166323_G | chr19:45589595_C |
| chr11:121451813_T | chr19:1013634_T | chr19:45728059_C |
| chr14:53400629_C | chr19:41098691_C | chr19:51681965_A |
| chr14:92993336_C | chr19:45097027_A | chr20:55018260_C |

All publications and patents mentioned in the above specification are herein incorporated by reference in their entirety for all purposes. Various modifications and variations of the described compositions, methods, and uses of the technology will be apparent to those skilled in the art without departing from the scope and spirit of the technology as described. Although the technology has been described in connection with specific exemplary embodiments, it should be understood that the technology as claimed should not be unduly limited to such specific embodiments. Indeed, various modifications of the described modes for carrying out the technology that are obvious to those skilled in the art are intended to be within the scope of the following claims.

What is claimed is:

1. A method for characterizing a plurality of neurodegenerative pathological features of a cognitive impairment in a human subject, comprising:
(a) detecting, in a sample obtained from the subject, a status of first markers in a first panel of markers or markers in linkage disequilibrium with markers in the first panel of markers, wherein the first panel of markers is associated with a first neurodegenerative pathological feature of the cognitive impairment;
(b) detecting, in the same sample obtained from the subject, a status of second markers in a second panel of markers or markers in linkage disequilibrium with markers in the second panel of markers, wherein the second panel of markers is associated with a second neurodegenerative pathological feature of the cognitive impairment; and
(c) characterizing a presence or risk of the first and second neurodegenerative pathological features of the cognitive impairment in the subject based on the status of the first markers and the status of the second markers.

2. A method of selecting a patient for participation in a clinical trial, comprising:
characterizing a plurality of neurodegenerative pathological features of a cognitive impairment in a human subject according to claim 1; and
selecting the patient for participation in the clinical trial based on the characterized presence or risk of the first and second neurodegenerative pathological features of the cognitive impairment in the subject.

3. The method of claim 1 or 2, wherein characterizing the risk of the first and second neurodegenerative pathological features in the subject comprises characterizing a risk that the subject had at the time the sample was obtained from the subject the first neurodegenerative pathological feature, the second neurodegenerative pathological feature, or both.

4. The method of any one of claims 1-3, wherein characterizing the risk of the first and second neurodegenerative pathological features in the subject comprises characterizing a risk that the subject will develop the first neurodegenerative pathological feature, the second neurodegenerative pathological feature, or both.

5. The method of any one of claims 1-4, wherein characterizing the risk of the first and second neurodegenerative pathological features in the subject comprises characterizing a risk that the subject had at the time the sample was obtained from the subject or that the subject will develop the first neurodegenerative pathological feature, the second neurodegenerative pathological feature, or both.

6. The method of any one of claims 1-5, wherein characterizing a risk of the first and second neurodegenerative pathological features in the subject comprises separately characterizing (i) the risk of the first neurodegenerative feature based on the status of the first markers, and (ii) the risk of the second neurodegenerative feature in the subject based on the status of the second markers.

7. The method of any one of claims 1-6, wherein characterizing a risk of the first and second neurodegenerative pathological features in the subject comprises characterizing a composite risk of the first neurodegenerative feature and the second neurodegenerative feature in the subject.

8. The method any one of claims 1-7, wherein characterizing a risk of the first and second neurodegenerative pathological features in the subject comprises characterizing a composite risk of the first neurodegenerative feature or the second neurodegenerative feature in the subject.

9. The method of any one of claims 1-8, wherein detecting a status of first markers or a status of second markers comprises determining the presences or absence of the first markers or the presence or absence of the second markers.

10. The method of any one of claims 1-9, wherein the presence or risk of the first neurodegenerative pathological feature and the presence or risk of the second neurodegenerative pathological feature are characterized using independently selected machine learning systems.

11. The method of any one of claims 1-10, comprising characterizing a presence or risk of three or more neurodegenerative pathological features of the cognitive impairment in the subject using independently selected machine learning systems.

12. The method of any one of claims 1-11, wherein the first neurodegenerative pathological feature and/or the second neurodegenerative pathological feature is amyloid beta, Lewy bodies, tau protein, cerebral amyloid angiopathy (CAA), or a progression of the cognitive impairment.

13. The method of any one of claims 1-12, wherein the first markers and/or the second markers comprise one or more genetic markers.

14. The method of claim 13, wherein the one or more genetic markers comprise one or more functional SNPs and/or one or more tag SNPs.

15. The method of claim 13 or 14, wherein the one or genetic markers comprise one or more of a DNA structural variant, a DNA copy number, a DNA repeat expansion, a DNA short tandem repeat (STR), DNA deletion 20 bases in length or less, a DNA deletion more than 21 bases in length, a DNA insertion, an RNA expression level, an RNA SNP, an RNA fusion, an RNA splice variant, or a DNA methylation status.

16. The method of any one of claims 1-15, wherein the first markers and/or the second markers comprise clinical markers and/or therapeutic markers.

17. The method of any one of claims 1-16, wherein said markers comprise an APOE allele 2 copy number, APOE allele 4 copy number, biological sex, and/or age.

18. The method of any one of claims 1-17, wherein characterizing the presence or risk of the first and second neurodegenerative pathological features of the cognitive impairment in the subject comprises inputting data describing the status of the first set of markers and/or the second set of markers into one or more machine learning systems.

19. The method of claim 18, wherein the one or more machine learning systems output a predictor of the presence or risk of the first neurodegenerative pathological feature and the presence or risk of the second neurodegenerative pathological feature.

20. The method of any one of claims 1-19, wherein at least the first neurodegenerative pathological feature and the second neurodegenerative pathological feature are used to enroll the subject in a clinical trial.

21. The method of any one of claims 1-20, wherein at least the first neurodegenerative pathological feature and the second neurodegenerative pathological feature are used to determine a course of a treatment for the cognitive impairment.

22. The method of any one of claims 1-21, wherein detecting the status of one or more markers among the first markers or the second markers comprises sequencing nucleic acids from the sample.

23. A method for characterizing a human subject as having or at risk for a cognitive impairment, the method comprising:
(a) detecting, in a sample obtained from the subject, the status of markers in a panel of markers or markers in linkage disequilibrium with the markers in the panel of markers; and
(b) characterizing the presence or risk of a cognitive impairment in the subject based on the status of said markers of said panel of markers.

24. A method of selecting a patient for participation in a clinical trial, comprising:
characterizing the human subject as having a cognitive impairment according to the method of claim 23; and
selecting the patient for participation in the clinical trial based on the characterized presence or risk of the cognitive impairment in the subject.

25. The method of claim 23 or 24, wherein characterizing the presence or risk of a cognitive impairment in the subject comprising characterizing the risk that the subject had the cognitive impairment at the time the sample was obtained from the subject.

26. The method of any one of claims 23-25, wherein characterizing the presence or risk of a cognitive impairment in the subject comprising characterizing the risk that the subject will develop the cognitive impairment.

27. The method of any one of claims 22-26, wherein characterizing the presence or risk of a cognitive impairment in the subject comprising characterizing the risk that the subject had, at the time the sample was obtained from the subject, or that the subject will develop the cognitive impairment.

28. The method of any one of claims 22-27, wherein detecting the status of markers comprises determining the presence or absence of the markers

29. The method of any one of claims 22-28, wherein characterizing the presence or risk of a cognitive impairment comprises predicting the presence of a neurodegenerative pathological feature.

30. A method for characterizing a sample as having been obtained from a human subject having cognitive impairment, the method comprising:
(a) receiving a sample obtained from the subject;
(b) detecting, in a sample obtained from the subject, the presence or absence one or more markers of cognitive impairment selected from a panel of markers or markers in linkage disequilibrium with the markers;
(c) using a machine learning system to receive data generated in steps (b) and output a cognitive impairment risk assessment for the human subject from which the sample was obtained; and
(d) characterizing the subject as having a cognitive impairment or having an increased risk of cognitive impairment based on the risk assessment of step (c).

31. The method of claim 30, further comprising identifying said subject as a candidate for a clinical trial.

32. The method of claim 30 or 31, wherein characterizing the subject as having a cognitive impairment or having an increased risk of cognitive impairment comprises predicting the presence of a neurodegenerative pathological feature.

33. A method of testing a subject for cognitive impairment, the method comprising:
(a) obtaining a sample from the subject;
(b) providing the sample to a testing facility to be tested for the presence or absence of markers for a panel or markers in linkage disequilibrium with the markers; and
(c) receiving a report from the testing facility indicating presence or risk of cognitive impairment in the subject.

34. A method for characterizing a human subject as having a cognitive impairment, the method comprising:
(a) detecting, in a sample obtained from the subject, the presence or absence of markers for a panel of markers selected from the markers provided by Table 2 or markers in linkage disequilibrium with the markers in Table 2, and
(b) characterizing the presence or risk of cognitive impairment in the subject based on the presence or absence of said markers of said panel of markers.

35. The method of claim 34, wherein the human subject is suspected of suffering from a cognitive disorder based on the presence of symptoms of a cognitive disorder.

36. The method of claim 34 or 35, wherein the human subject is suspected of suffering from a cognitive disorder based on an assessment of cognitive ability.

37. The method of claim 36, wherein the human subject is suspected of suffering from a cognitive disorder based on a change with time of a score from an assessment of cognitive ability.

38. The method of any one of claims 34-37, wherein characterizing the presence or risk of cognitive impairment in the subject comprises inputting data describing the presence or absence of said markers of said panel of markers into a machine learning system.

39. A method for classifying progression of cognitive impairment in a human subject, the method comprising:
(a) detecting, in a sample obtained from the subject, the status of markers in a panel of markers or markers in linkage disequilibrium with the markers in the panel of markers; and
(b) classifying progression of cognitive impairment human subject based on the status of said markers of said panel of markers.

40. A method for classifying progression of cognitive impairment in a human subject, the method comprising:
(a) detecting, in a sample obtained from the subject, the presence or absence of markers for a panel of markers selected from the markers provided by Table 1 or markers in linkage disequilibrium with the markers in Table 1; and
(b) classifying progression of cognitive impairment in the human subject based on the presence or absence of said markers of said panel of markers.

41. A method for characterizing a sample as having been obtained from a human subject having cognitive impairment, comprising:
(a) receiving a sample obtained from the subject;
(b) generating input data by detecting, in the sample obtained from the subject, the status of a plurality of markers of cognitive impairment;
(c) characterizing a risk for cognitive impairment for the subject using a trained machine learning model configured to receive the generated data and output a cognitive impairment risk assessment for the subject, the trained machine learning model comprising:
(i) a plurality of parameters identified using a training data set comprising, for each training sample in the training data set, a status of one or more markers of cognitive impairment and a cognitive impairment status of a subject associated with the training sample; and
(ii) a function representing the relation between the status of the one or more markers of cognitive impairment and the cognitive impairment risk assessment; and
(d) generating a report characterizing the sample as having been obtained from a human subject having cognitive impairment or having an increased risk of cognitive impairment based on the outputted cognitive impairment risk assessment.

42. A method for characterizing a sample as having been obtained from a human subject having cognitive impairment, the method comprising:
(a) receiving a sample obtained from the subject;
(b) detecting, in a sample obtained from the subject, the presence or absence of a first marker of cognitive impairment selected from the markers provided by Table 2 or in linkage disequilibrium with a marker provided by Table 2;
(c) detecting, in said sample, the presence or absence of a second marker of cognitive impairment selected from the markers provided by Table 2 or in linkage disequilibrium with a marker provided by Table 2;
(d) using a machine teaming system to receive data generated in steps (b) and (c) and output a cognitive impairment risk assessment for the human subject from which the sample was obtained; and
(e) generating a report characterizing the sample as having been obtained from a human subject having cognitive impairment or having an increased risk of cognitive impairment based on the risk assessment of step (d).

43. A method for classifying progression of cognitive impairment in a human subject, the method comprising:
(a) receiving a sample obtained from the subject;
(b) detecting, in a sample obtained from the subject, the presence or absence of a first marker of cognitive impairment selected from the markers provided by Table 1 or in linkage disequilibrium with a marker provided by Table 1;
(c) detecting, in said sample, the presence or absence of a second marker of cognitive impairment selected from the markers provided by Table 1 or in linkage disequilibrium with a marker provided by Table 1;
(d) using a machine learning system to receive data generated in steps (b) and (c) and output a cognitive impairment progression classifier for the human subject from which the sample was obtained; and
(e) generating a report classifying the progression of cognitive impairment in the human subject based on the risk assessment of step (d).

44. The method of any one of claims 41-43, further comprising identifying said subject as a candidate for a clinical trial.

45. A method of testing a subject for cognitive impairment, the method comprising:
(a) obtaining a sample from the subject;
(b) providing the sample to testing facility to be tested for the presence or absence of markers for a panel of markers selected from the markers provided by Table 2 or markers in linkage disequilibrium with the markers in Table 2; and
(c) receiving a report from the testing facility indicating presence or risk of cognitive impairment in the subject.

46. A method of classifying progression of cognitive impairment in a human subject, the method comprising:
(a) obtaining a sample from the subject;
(b) providing the sample to testing facility to be tested for the presence or absence of markers for a panel of markers selected from the markers provided by Table 1 or markers in linkage disequilibrium with the markers in Table 1; and
(c) receiving a report from the testing facility classifying progression of cognitive impairment in the human subject.

47. A method for characterizing plurality of neurodegenerative pathological features of a cognitive impairment in a human subject, comprising:
(a) generating first input data by detecting, in a sample obtained from the subject a status of markers in a first panel of markers or markers in linkage disequilibrium with markers in the first panel of markers, wherein the first panel of markers is associated with a first neurodegenerative pathological feature of the cognitive impairment;
(b) characterizing a risk for the first neurodegenerative pathological feature for the subject using a first trained machine learning model configured to receive the generated first input data and output a risk assessment for the first neurodegenerative pathological feature for the subject, the first trained machine learning model comprising:
(i) a plurality of parameters identified using a first training data set comprising, for each training sample in the first training data set, a status of one or more markers of the first neurodegenerative pathological feature and a first neurodegenerative pathological feature status of a subject associated with the training sample; and
(ii) a function representing the relation between the status of the one or more markers of the first neurodegenerative pathological feature and the risk of the first neurodegenerative pathological feature;
(c) generating second input data by detecting, in the sample obtained from the subject a status of markers in a second panel of markers or markers in linkage disequilibrium with markers in the second panel of markers, wherein the second panel of markers is associated with a second neurodegenerative pathological feature of the cognitive impairment;
(d) characterizing a risk for the second neurodegenerative pathological feature for the subject using a second trained machine learning model configured to receive the generated second input data and output a risk assessment for the second neurodegenerative pathological feature for the subject, the second trained machine learning model comprising:
(i) a plurality of parameters identified using a second training data set comprising, for each training sample in the second training data set, a status of one or more markers of the second neurodegenerative pathological feature and a second neurodegenerative pathological feature status of a subject associated with the training sample; and
(ii) a function representing the relation between the status of the one or more markers of the second neurodegenerative pathological feature and the risk of the second neurodegenerative pathological feature; and
(e) generating a report characterizing the risk of the first neurodegenerative pathological feature and the second neurodegenerative pathological feature based on the output from the first trained machine learning model and the second trained machine learning model.

48. A method of selecting a patient for participation in a clinical trial, comprising:
characterizing a plurality of neurodegenerative pathological features of a cognitive impairment in a human subject according to claim 47; and
selecting the patient for participation in the clinical trial based on the characterized risk of the first and second neurodegenerative pathological features of the cognitive impairment in the subject.

49. The method of claim 47 or 48, wherein characterizing the risk of the first neurodegenerative pathological feature and the second neurodegenerative pathological feature comprises characterizing a risk that the subject had at the time the sample was obtained from the subject the first neurodegenerative pathological feature, the second neurodegenerative pathological feature, or both.

50. The method of any one of claims 47-49, wherein characterizing the risk of the first neurodegenerative pathological feature and the second neurodegenerative pathological feature comprises characterizing a risk that the subject will develop the first neurodegenerative pathological feature, the second neurodegenerative pathological feature, or both.

51. The method of any one of claims 47-50, wherein characterizing the risk of the first neurodegenerative pathological feature and the second neurodegenerative pathological feature comprises characterizing a risk that the subject had at the time the sample was obtained from the subject or that the subject will develop the first neurodegenerative pathological feature, the second neurodegenerative pathological feature, or both.

52. The method of any one of claims 47-51, wherein characterizing the risk of the first neurodegenerative pathological feature and the second neurodegenerative pathological feature comprises characterizing a composite risk of the first neurodegenerative feature and the second neurodegenerative feature in the subject.

53. The method any one of claims 47-52, wherein characterizing the risk of the first neurodegenerative pathological feature and the second neurodegenerative pathological feature comprises characterizing a composite risk of the first neurodegenerative feature or the second neurodegenerative feature in the subject.

54. The method of any one of claims 47-53, wherein detecting the status of markers in the first panel or the status of markers in the second panel comprises determining the presences or absence of the markers in the first panel or the presence or absence of markers in the second panel.

55. The method of any one of claims 47-54, wherein first machine learning model and the second machine learning model are independently selected.

56. The method of any one of claims 47-55, comprising characterizing a risk of three or more neurodegenerative pathological features of the cognitive impairment in the subject using independently selected machine learning systems.

57. The method of any one of claims 47-56, wherein the first neurodegenerative pathological feature and/or the second neurodegenerative pathological feature is amyloid beta, Lewy bodies, tau protein, cerebral amyloid angiopathy (CAA), or a progression of the cognitive impairment.

58. The method of any one of claims 47-57, wherein the markers of the first panel and/or the markers of the second panel comprise one or more genetic markers.

59. The method of claim 58, wherein the one or more genetic markers comprise one or more functional SNPs and/or one or more tag SNPs.

60. The method of any one of claims 47-59, wherein the first markers and/or the second markers comprise clinical markers and/or therapeutic markers.

61. The method of any one of claims 47-60, wherein said markers comprise an APOE allele 2 copy number, APOE allele 4 copy number, biological sex, and/or age.

62. The method of any one of claims 47-61, further comprising enrolling the subject in a clinical trial based on the risk of the first neurodegenerative pathological feature and the second neurodegenerative pathological feature.

63. The method of any one of claims 47-62, wherein at least the first neurodegenerative pathological feature and the second neurodegenerative pathological feature are used to determine a course of a treatment for the cognitive impairment.

64. The method of any one of claims 47-63, wherein detecting the status of one or more markers among the first markers or the second markers comprises sequencing nucleic acids from the sample.

65. The method of any one of claims 1-64, wherein the cognitive impairment is associated with Alzheimer's disease or dementia.
